(19) United States
(12) Patent Application Publication
(10) Pub. No.: US 2006/0120962 A1
(43) Pub. Date: Jun. 8, 2006

(54) Cardiac safe, rapid medication delivery

(76) Inventors: Joshua D. Rabinowitz, Princeton, NJ (US); Peter M. Lloyd, Walnut Creek, CA (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 11/248,598

(22) Filed: Oct. 12, 2005

Related U.S. Application Data
(60) Provisional application No. 60/618,243, filed on Oct. 12, 2004.
(60) Provisional application No. 60/712,760, filed on Aug. 30, 2005.

(51) Int. Cl. A61K 49/00; A61K 9/14; A61L 9/04

(52) U.S. Cl. 424/9.2; 424/46

(57) ABSTRACT

The disclosure provides methods and compositions for providing an effective dose of an active agent and/or drug composition to a subject by inhalation. The methods of the disclosure are useful in determining a maximal effective dose that limits cardiovascular damage upon inhalation.

### CROSS-REFERENCE TO RELATED APPLICATIONS

This application claims the benefit under 35 U.S.C. § 119 of U.S. Provisional Patent Application Ser. No. 60/618,243, entitled “Cardiac Safe, Rapid Medication Delivery,” filed Oct. 12, 2004 and U.S. Provisional Patent Application Ser. No. 60/712,760, entitled “Cardiac Safe, Rapid Medication Delivery,” filed Aug. 30, 2005.

### TECHNICAL FIELD

This invention relates to determining dosages, and more particularly to methods for determining safe and effective dosages of drug active agents for rapid systemic delivery via inhalation and inhalation devices comprising a dosage determined by the methods of the invention.

### BACKGROUND

For certain drugs, rapid entry into the circulation of a subject is important. This importance can arise, for example, from a desire of the subject for rapid symptom alleviation. Speed of delivery determines not only the time of onset of drug action, but also the peak drug concentrations obtained in the body, with more rapid delivery resulting in higher peak drug concentrations. For many drugs, achieving appropriate peak drug concentrations is critical, because peak concentrations that are too low can result in failure of the drug to be effective, and peak concentrations that are too high can result in unwanted side effects. Such side effects can be particularly serious when they involve undesirably high peak drug concentrations in the cardiac circulation, as the resulting adverse events may then include myocardial infarction (heart attack) or cardiac arrhythmia (abnormal heart rhythm).

### SUMMARY

The invention provides a method of identifying a cardiovascular safe dose of an inhaled drug active agent. The method includes (a) determining a peak arterial plasma concentration (or peak left ventricular concentration) of the drug active agent following (i) inhalation, and (ii) intravenous delivery of the drug active agent, (b) identifying a cardiovascular safe intravenous dose of the drug active agent based on cardiovascular safety measurements taken after intravenous delivery of the drug active agent; and (c) defining a cardiovascular safe inhaled dose of the drug active agent as less than or equal to the cardiovascular safe intravenous dose divided by the ratio of the peak plasma drug concentration produced by inhalation delivery relative to that produced by intravenous delivery.

The invention also provides a method of identifying a cardiovascular safe dose of a drug active agent for systemic delivery by inhalation. The method includes (a) determining a rate of absorption of the drug active agent into the arterial circulation (or left ventricle of the heart) when delivered by inhalation, and (b) determining the cardiovascular safety of the same drug active agent delivered at a substantially identical rate at one or more doses by an intravenous route, wherein the cardiovascular safe dose of the inhaled active agent is equal to or less than a dose that is determined to be safe when delivered at a substantially identical rate by the intravenous route.

The invention further provides a method of delivering a drug active agent to a mammal. The method includes administering by inhalation the drug active agent in the form of an aerosol, wherein the administration produces a spike index between 2 and 6, and wherein the peak plasma concentration of the drug active agent in the left ventricle of the heart is achieved in less than 30 seconds.

The invention provides a method of establishing an appropriate dose of an inhaled drug active agent. The method includes administering to a mammal a substantially identical amount of the drug active agent by both inhalation and intravenous (IV) injection; identifying an inhaled peak plasma concentration in the arterial circulation (or left ventricle of the heart) upon delivery of the drug active agent by inhalation; identifying an IV peak plasma concentration in the arterial circulation (or left ventricle of the heart) upon delivery of the composition by IV injection; calculating a spike index for the inhalation delivery; identifying an intravenous dose of the drug active agent that is safe to deliver to the mammal, and dividing this dose by the spike index to yield a safe inhalation dose; identifying an intravenous dose of the drug active agent that produces a desirable response in the mammal, and dividing this dose by the spike index to yield an effective inhalation dose, wherein the effective inhalation dose is less than the safe inhalation dose; and selecting the appropriate dose of the drug active agent, wherein the dose is less than or equal to the safe inhalation dose and greater than or equal to the effective inhalation dose.

The invention also provides an inhalation device comprising a dosage determined by the methods of the invention. In one aspect, the invention provides an aerosol-releasing device for inhalation therapy, wherein the device releases one or more doses of aerosol that, when inhaled by a mammal, result in a spike index of between about 1.5 and 10, or between 2 and 6.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph showing the left ventricle plasma concentration versus time of prochlorperazine when delivered to a subject by inhalation and IV bolus.

FIG. 2 is a graph showing the venous plasma concentration versus time of prochlorperazine when delivered to a subject by inhalation and IV bolus.

FIG. 3 is a graph showing the left ventricle plasma concentration versus time of alprazolam when delivered to a subject by inhalation and IV bolus.

FIG. 4 is a graph showing the venous plasma concentration versus time of alprazolam when delivered to a subject by inhalation and IV bolus.

FIG. 5 is a schematic of an aerosol generation and administration system of the type used in the Examples 1 and 2.

### DETAILED DESCRIPTION

It is to be understood that both the foregoing general description and the following detailed description are exemplary and explanatory only and are not restrictive of the invention, as claimed. It must be noted that as used herein and in the appended claims, the singular forms “a,” “and,” and “the” include plural referents unless the context clearly dictates otherwise. Thus, for example, reference to “a drug” includes mixtures of different drugs, reference to “an agent” refers to one or more agents, and so forth. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

The term “aerosol” refers to a suspension of solid or liquid particles in a gas. Exemplary non-limiting aerosol preparations suitable for administration by inhalation to a subject include, but are not limited to, pure liquid droplets, solutions in liquid droplet form and solids in powder form. In certain embodiments, an aerosol preparation can include a pharmaceutically acceptable carrier, excipient and/or surfactant. For example, a pharmaceutically acceptable carriers include an inert compressed gas, e.g., nitrogen, or inactive solid particles, e.g., lactose particles.

The term “administering by inhalation” refers to the administration of a composition to a subject in aerosol form such that the subject inhales the composition into the pulmonary tract, whether by mouth, through an endotracheal tube, etc.

The term “drug active agent” means any substance that is used in the prevention, diagnosis, alleviation, treatment or cure of a condition. The terms “drug”, “drug composition” and “drug active agent” are used interchangeably herein. It is noted that a drug active agent may include carriers, excipients, surfactants, etc.

The term “drug degradation product” or “thermal degradation product” are used interchangeably and means any byproduct, which results from heating the drug(s) and is not responsible for producing a therapeutic effect.

The term “intravenous,” when used herein to describe the site of an injection or to intravenous delivery of a drug active agent, refers to an injection within a peripheral vein of a mammal, such as a saphenous vein, basilic vein, cephalic vein, dorsal venous arch or dorsal metacarpal vein of the hand, or dorsal venous arch or dorsal metatarsal vein of the foot. It does not refer to an injection within a central vein, such as a jugular, bracheocephalic, or subclavian vein or the superior or inferior vena cava.

The term “intravenous bolus,” when used herein, refers to a rapid intravenous injection having a duration, unless otherwise specified, of about 5 seconds.

The term “thermal stability ratio” or “TSR” means the % purity/(100%−% purity) if the % purity is <99.9%, and 1000 if the % purity is ≧99.9%. For example, a respiratory drug vaporizing at 90% purity would have a TSR of 9. A “heat stable drug” refers to a drug that has a TSR≧9 when vaporized from a film of some thickness between 0.05 μm and 20 μm. A determination of whether a drug classifies as a heat stable drug can be made as described in Example 4.

The term “peak plasma concentration” refers to the maximum level of a drug active agent in the plasma of a subject after initiation of administration of the drug active agent to the subject.

The term “peak arterial plasma concentration” refers to the peak plasma concentration in arterial plasma (where “arterial” refers to that portion of the circulatory system extending from the left heart (atrium and ventricle) to the aorta and its arterial branches such as the coronary, carotid, subclavian, femoral, brachial radial, and ulnar arteries, terminating in (but excluding) arterioles and capillaries).

The term “peak left ventricular plasma concentration” refers to the peak plasma concentration in left ventricular plasma (where “left ventricular” refers to that portion of the circulatory system extending from the mitral valve to the aortic valve, and thereby refers to a subset of the arterial system). In certain cases, when attempting to obtain left ventricular samples, passage of a catheter through the aortic valve proves technically challenging; in such cases samples obtained in the aorta within a few centimeters of the aortic valve may be considered “left ventricular” samples for pharmacokinetic purposes.

The term “peak venous plasma concentration refers to the peak plasma concentration in the venous plasma (where “venous” refers to that portion of the circulatory system beginning with small veins (i.e., excluding venules and capillaries) which branch from larger veins which in turn lead to either the superior or inferior vena cava and the right heart (atrium and ventricle).

The term “purity” as used herein with respect to the aerosol purity refers to the fraction of drug composition in the aerosol/the fraction of drug composition in the aerosol plus drug degradation products. Thus, purity is relative with regard to the purity of the starting material. For example, when the starting drug or drug composition used for substrate coating contained detectable impurities, the reported purity of the aerosol does not include those impurities present in the starting material that were also found in the aerosol, e.g., in certain cases if the starting material contained a 1% impurity and the aerosol was found to contain the identical 1% impurity, the aerosol purity may nevertheless be reported as >99% pure, reflecting the fact that the detectable 1% purity was not produced during the vaporization-condensation aerosol generation process.

A “spike index” of a drug active agent refers to the peak arterial plasma concentration (or peak left ventricular plasma concentration) (Cₘₐₓ) produced by delivery of the drug active agent to a subject through a non-intravenous delivery route (e.g., by inhalation), divided by the peak plasma concentration produced upon delivery of the same drug active agent in a substantially identical amount by an intravenous injection over a specified duration between 0 and 300 seconds. For example, a “spike index-120” is a spike index calculated based upon an intravenous injection occurring over a duration of approximately 120 seconds. When not otherwise specified, the term spike index refers to a spike index-120. In the case where the amount of drug active agent delivered by the two routes differs substantially (e.g., by more than 10% or 20%) the spike index may be calculated by correcting for the difference in doses as follows:
Spike Index=(Cₘₐₓtest/CₘₐₓIV)/(Doseₜₑₛₜ/Dose_{IV})]
The “spike index” is a unit-less parameter obtained from the ratio of two measurements having identical units. To obtain a dose based upon the spike index, the spike index is used as the denominator with the numerator being, for example, a safe effective dose or a therapeutic dose. Typically the dose of the numerator will be in μg or mg.

The term “therapeutic systemic concentration” refers to the concentration of a drug active agent within the bloodstream of a subject at which a desired effect (e.g., a therapeutic effect) of the drug is achieved.

The term “thermal vapor” refers to an aerosol, to a vapor phase, or to a mixture of an aerosol and a vapor phase. In certain embodiments, the thermal vapor is formed by heating. In certain embodiments, the thermal vapor comprises a drug active agent. In certain embodiments, the thermal vapor comprises a drug active agent and a carrier. The term “vapor phase” refers to a gaseous phase.

Drug compounds and active agents can be delivered to a mammalian body through a variety of routes including injection, oral intake, and inhalation. For rapid delivery, deep lung inhalation has a number of key advantages, including the large absorptive surface area (˜100 m²) of the deep lung (alveoli), the thickness and permeability of the barrier separating the alveolar airspace from the pulmonary capillary bed, and the direct passage of absorbed drug and active ingredients from the pulmonary circulation to the left heart and from there to the arterial circulation. By generation of appropriate aerosol particles as described herein, agents inhaled as aerosol particles may reach the body and brain in less than a minute. Some characteristics associated with aerosol particles that rapidly release drug into the left heart and arterial circulation upon inhalation delivery are the following: (1) appropriate size to reach and deposit in alveoli (e.g., about 1-3 μm diameter), (2) rapid dissolution in the lung, and (3) ready passage of the drug and/or active ingredient from the alveoli to the bloodstream, driven for maximally rapid absorption ideally by as large as possible of a concentration gradient between the deposition site in the lung and the bloodstream.

Thus, the inhalation route can be used to rapidly deliver a drug active agent into systemic circulation. Certain drugs when inhaled as aerosols of appropriate particle size pass smoothly from the mouth and upper respiratory tract into the deep lung, where they deposit on alveolar tissue. From the alveoli, most small molecule drugs are well absorbed into the systemic circulation if delivered in an appropriate physical form (e.g., as pure drug in amorphous form, or as a concentrated drug solution), passing from the alveoli to the pulmonary capillary blood to the pulmonary vein, the left atrium, the left ventricle, and ultimately into the aorta and systemic arterial circulation.

The speed of pulmonary drug absorption depends primarily on the depth of penetration of the drug into the lung, the rate of dissolution of the delivered particles upon contact with the pulmonary surface, and the magnitude of the drug concentration gradient established between the lung surface and the blood stream. Maximally rapid absorption is enabled by delivery to the alveoli of fast-dissolving, pure drug particles. One means of producing particles that are very rapidly absorbed is thermal aerosol generation, including thermal generation of drug aerosols devoid of excipients, organic solvents, and propellants. Methods and devices for providing drug-containing thermal aerosols are disclosed in, e.g., “Acute treatment of headache with phenothiazine antipsychotics,” U.S. patent application Ser. No. 10/719,763 (Publication No. US 2004-20040101481-A1), incorporated herein by reference in its entirety.

One typical feature of a thermal aerosol is the aerosol particle size (MMAD generally about 1 to 3 μm due to mixing of vapor drug into cooling air) and associated deposition characteristics (alveolar). Desirable deposition characteristics of thermal aerosols produced using the delivery devices of the present invention typically includes slow aerosol velocity (which avoids throat impaction) which can result from not using a propellant to generate the aerosols, and rapid generation (with most of the drug aerosol in the first liter of inspired air due to rapid heating of drug). Another typical feature of a thermal aerosol is amorphous particle nature, which is often liquid or otherwise highly disordered solid (due to condensation of molecularly disperse drug without time for substantial solid organization or crystal formation), and associated rapid dissolution characteristics. Another typical feature of a thermal aerosol is a high drug concentration in the aerosol (due to ability to make aerosol particles of >90% pure drug; the aerosol can be free of solvent, excipients, propellants, etc.)

Inhalation of a drug active agent in the form of a thermal aerosol can result in peak arterial plasma concentration (or peak left ventricular plasma concentration) in less than 15, 20, 30, 45, or 60 seconds. These peak arterial plasma concentrations (or peak left ventricular plasma concentrations) typically occur more rapidly even than after IV bolus injection. Peak plasma concentrations in the venous circulation may also occur more rapidly than after IV bolus injection. Inhalation of aerosols yielding rapid absorption can beneficially result in rapid achievement of substantial systemic levels of a desired drug. Such rapid absorption may be advantageous when rapid onset of drug action and/or high peak drug concentrations are desired, but only if the peak drug concentrations can be adequately controlled to avoid unwanted side effects.

The invention addresses this need by providing methods and compositions for obtaining desirable concentrations of a drug in the left heart and arterial circulation of a subject via inhalation, with particular techniques for rapidly achieving high but safe peak drug concentrations.

Thus, the invention provides a method of rapidly achieving desirable concentrations of a drug active agent in the arterial circulation (or left ventricle of the heart) of a subject. A subject can be any mammal including, but not limited to, canines, bovines, equines, felines, porcine species, primates, and humans.

The method involves a subject inhaling a drug active agent in aerosol form, wherein a desirable peak plasma concentration in the arterial circulation (or left ventricle of the heart) of the mammal is reached within 60 seconds of inhalation of the drug active agent, and typically a desirable peak plasma concentration is reached within 15 or 20 or 30 to 45 seconds of inhalation of the drug active agent. The inhaled peak plasma concentration is desirable in that it is quite high, especially in the arterial circulation (or left ventricle of the heart), substantially exceeding the concentration achieved following oral delivery of the same dose of the drug active agent or even following delivery of the same dose of the drug active agent as an intravenous injection given over about 2 minutes. The inhaled peak plasma concentration in the arterial circulation (or left ventricle of the heart) is substantially greater (e.g., 1.5, 2, 4, 6, or 8-fold greater) than the peak plasma concentration in the venous circulation, which is advantageous especially in the case where very brief arterial action of the drug is desired (e.g., for terminating a cardiac arrhythmia); furthermore, the peak arterial plasma concentration (or peak left ventricular plasma concentration) occurs prior to the peak venous plasma concentration, generally by about 15 to 120 seconds, most commonly around 30, 45, or 60 seconds.

In one aspect of the invention, the inhaled peak plasma concentration can be characterized by a spike index of at least 1.5, 2, or even 2.5 (although a higher spike index is useful in some instances). Where the dose is measured in mg of drug delivered into the respiratory tract of the mammal, the inhaled peak plasma concentration measured in ng/mL can exceed 5, 10, 20, 50, 75, 100, 150, 200, 250, 300, 350 or 400 times the inhaled dose.

In nearly all cases, it is desirable to avoid peak plasma concentrations that are so high as to result in substantial acute toxicity or other side effects or adverse events. In particular, it is desirable to avoid peak plasma concentrations so high as to result in cardiac or cardiovascular adverse events.

In one aspect of the invention, the peak plasma concentration produced by inhalation is similar to or less than the peak plasma concentration produced by IV bolus delivery of a substantially identical dose of the drug active agent (over a bolus duration of less than or equal to 5 seconds). In another aspect, the peak plasma concentration produced by inhalation ranges from 0.5 to 1.5 times the peak plasma concentration produced by IV bolus delivery of a substantially identical dose of the drug active agent. Typically, the spike index of a drug active agent delivered by inhalation is less than 10, 8, 6, or 4. In one aspect of the invention, the peak arterial plasma concentration (or peak left ventricular plasma concentration) does not exceed the peak venous plasma concentration by greater than 10, 15, or 20-fold. Where the dose is measured in mg of drug delivered into the respiratory tract of the subject, the peak plasma concentration measured in ng/mL is typically less than or equal to about 2000, 1500, 1200, 1000 or 750 times the inhaled dose.

In one aspect, the invention provides a method whereby an appropriate non-intravenous delivered dose can be determined for a particular form of a drug, where data on the IV form of the same drug exists. The method includes administering a dose of drug to a subject by a test route of administration (e.g., by inhalation), wherein the dose is selected to provide a measurable drug concentration in the arterial circulation (or left ventricle of the heart). In one aspect of the invention, the inhaled drug has a number concentration of at least 10⁷ particles/mL carrier gas. Preferably, the dose of drug administered does not cause any measurable side effect(s), or at least does not cause any severe side effect(s). The concentration of drug in the arterial circulation (or left ventricle of the heart) is then measured over a range of times (e.g., 15, 30, 60, 120, and 300 seconds) using known techniques to define the inhaled peak plasma concentration (Cₘₐₓtest).

A substantially similar or identical amount of drug is then administered by IV injection. The concentration of drug in the arterial circulation (or left ventricle of the heart) is then measured over a similar time or time period. The concentration of drug in the arterial circulation (or left ventricle of the heart) is defined as IV peak plasma concentration (CₘₐₓIV). In one aspect, the inhaled administration and IV administration are performed in the same subject following a return to baseline, undetectable or very low drug concentrations in the arterial circulation (or left ventricle of the heart) of that subject. In another aspect, different subjects are used, typically the same species and having similar age, weight, and the like. Furthermore, one of skill in the art will recognize that either the IV or the test route (e.g., inhaled route) of administration may occur first.

One of skill in the art will recognize that knowing the LD₅₀ of an IV injected dose of the drug will assist in calculating the proper doses to be used to avoid serious side effects associated with IV injection. Furthermore, the IV LD₅₀ information can be used to determine an initial dose range by a test route to avoid adverse events.

Using the Cₘₐₓtest and CₘₐₓIV values, the spike index can be determined. Once the spike index is calculated, an appropriate therapeutic dose for delivery by the test route of administration can be calculated by dividing the IV therapeutic dose by the spike index. For example, if the spike index of the test route was 2, and the therapeutic dose by the IV route was 5 mg, the appropriate therapeutic dose by the test route would be 2.5 mg (5 mg divided by 2). A key advantage of this means of calculating the appropriate therapeutic dose of the new route of administration (e.g., inhalation) is ensuring cardiac safety; the calculated dose by the test route of administration will not result in peak arterial plasma concentrations (or peak left ventricular plasma concentrations) substantially exceeding those produced by the already tested therapeutic IV dose.

Thus, the invention provides a method of ensuring cardiovascular safety of an inhaled drug active agent that has previously been extensively studied by the IV route. In addition, the invention provides a method of determining the cardiovascular safety of an inhaled agent (or agent delivered by another route), even when the agent has not been extensively studied by the IV route previously. The method includes determining an arterial (or left ventricular) concentration of a drug active agent following (i) inhalation, and (ii) intravenous delivery of the drug active agent. Typically, sufficient concentrations are determined to measure reliably Cₘₐₓ by both routes and thus to calculate a spike index. In addition, the cardiovascular safety of the drug active agent delivered by the IV route (with the same injection duration as for the above pharmacokinetic measurements) is measured at minimally one dose, and typically at three or more doses. The cardiovascular safety measurement is generally conducted in dogs, typically telemetrized dogs, although other mammals may also be used. Typically, by testing multiple doses, it is possible to define a high dose that is cardiac safe. This may be either the highest dose tested, if none of the tested doses produces significant adverse events, or the highest of the tested doses that does not produce unacceptable cardiovascular or other adverse effects (e.g., does not produce unacceptable acute changes in heart rate or rhythm or blood pressure or other unacceptable changes on echocardiogram). A cardiac safe high inhalation dose with respect to acute cardiovascular adverse events can then be calculated as the cardiac safe high IV dose (experimentally determined by the above safety measurements) divided by the ratio of the maximum drug concentration measured in the inhalation pharmacokinetic study to the maximum drug concentration measured in the IV pharmacokinetic study (e.g., the spike index). Typically, when the pharmacokinetic measurements are adequate to calculate a spike index, the cardiac safe high inhalation dose is the cardiac safe high IV dose divided by the spike index. Generally, when the experimental cardiac safety measurement are conducted in dogs or some other non-human species, human testing of the inhalation form of the drug active agent is initiated at substantially below the cardiac safe high inhalation dose, e.g., 2, 5, or 10 or more times below this dose.

Using the methodology of the invention, an appropriate dose of an inhaled drug active agent can be determined. The method includes administering to a mammal a substantially identical amount of a drug active agent by both inhalation and intravenous (IV) injection. The inhaled peak arterial plasma concentration (or peak left ventricular plasma concentration) is determined for both the inhaled and IV administered drug active agents. A spike index for the inhaled dose is then calculated. A cardiac safe inhalation dose of the drug active agent is obtained by dividing the cardiac safe high intravenous dose by the spike index. An effective inhalation dose can also be calculated by dividing an intravenous dose of the composition that produces a desirable response in a mammal by the spike index. Typically the effective inhalation dose is less than the cardiac safe inhalation dose. The desirable therapeutic dose will be less than or equal to the cardiac safe inhalation dose and greater than or equal to the effective inhalation dose.

The invention also provides a method of delivering a drug active agent to a mammal, the method comprising administering the drug active agent by inhalation in the form of an aerosol, wherein the administration produces a spike index between 2 and 6, and wherein the peak plasma concentration of the drug in the arterial circulation (or left ventricle of the heart) is achieved in less than 15, 20, 30, 45, or 60 seconds.

Any inhalation device can be used to deliver the drug active agent so long as the device is capable of providing an aerosol or other formulation to the bronchial, airway, and preferably the deep lung alveoli, with the aerosol particles preferably in a physical form where they dissolve and/or release drug rapidly upon deposition in the lung. One desirable breathing pattern for optimizing deep lung inhalation delivery involves a full exhalation, followed by a deep inhalation sometimes at a prescribed inhalation flow rate range, e.g., about 10 to about 150 liters/minute, followed by a breath hold of several seconds. In addition, ideally, the aerosol is not uniformly distributed in the air being inhaled, but is loaded into the early part of the breath as a bolus of aerosol, followed by a volume of clean air so that the aerosol is drawn into the alveoli and flushed out of the conductive airways, bronchi and trachea by the volume of clean air that follows. A typical deep adult human breath has a volume of about 2 to 5 liters. In order to ensure consistent delivery in the whole population of adult patients, delivery of the drug bolus should be completed in the first 1 liter or 1.5 liters or so of inhaled air.

In one aspect, the drug agent is vaporized in a minimum amount of time, typically no greater than 1 to 2 seconds to provide effective deep lung delivery.

In determining the proper dosage, studies involving intravenous (IV) injection of a drug active agent are often needed. The intravenous injection should be delivered smoothly over a specified duration. Typically an administration period of 120 seconds, or a bolus administration of 5 seconds, is used; however other time periods may be used (e.g., 1, 10, 20, 30, 60, 80, 100, 150, 180, 240, 300, or more seconds). The IV dose is typically administered by either steady manual injection over the full duration or by use of an infusion pump set to deliver the drug active agent at a steady rate. The solution for intravenous injection may use any suitable solvent system, e.g. water, aqueous buffer, ethanol, dimethylsulfoxide, propylene glycol, and the like, or mixtures thereof. The spike index may be measured in any suitable mammal species; typically in dogs, monkeys and/or humans. The arterial spike index refers to the spike index when the composition concentration is measured in the arterial blood, and the term ventricular spike index refers to the spike index when the composition concentration is measured in the left ventricular blood.

The drug active agents useful in the invention typically have a molecular weight in the range of about 150-700, typically in the range of about 200-650, more typically in the range of 250-600, still more typically in the range of about 250-500, and most typically in the range of about 300-450. Drug active agents in these weight ranges are particularly desirable for their facility of delivery as thermally generated aerosols, dissolution characteristics upon deposition in the lung, and ability to cross the membranes in the body such as the pulmonary-alveolar membrane. In some embodiments, drug active agents delivered as aerosols are heat stable. Preferably, drug active agents delivered by thermally generated aerosols are heat stable.

Specific drugs that can be used include, but are not limited to, drugs of one of the following classes: anesthetics, anticonvulsants, antidepressants, antidiabetic agents, antidotes, antiemetics, antihistamines, anti-infective agents, antineoplastics, antiparkisonian drugs, antirheumatic agents, antipsychotics, anxiolytics, appetite stimulants and suppressants, blood modifiers, cardiovascular agents, central nervous system stimulants, drugs for Alzheimer's disease management, drugs for cystic fibrosis management, diagnostics, dietary supplements, drugs for erectile dysfunction, gastrointestinal agents, hormones, drugs for the treatment of alcoholism, drugs for the treatment of addiction, immunosuppressives, mast cell stabilizers, migraine preparations, motion sickness products, drugs for multiple sclerosis management, muscle relaxants, nonsteroidal anti-inflammatories, opioids, other analgesics and stimulants, opthalmic preparations, osteoporosis preparations, prostaglandins, respiratory agents, sedatives and hypnotics, skin and mucous membrane agents, smoking cessation aids, Tourette's syndrome agents, urinary tract agents, and vertigo agents.

Typically, where the drug is an anesthetic, it is selected from one of the following compounds: ketamine and lidocaine.

Typically, where the drug is an anticonvulsant, it is selected from one of the following classes: GABA analogs, tiagabine, vigabatrin; barbiturates such as pentobarbital; benzodiazepines such as clonazepam; hydantoins such as phenytoin; phenyltriazines such as lamotrigine; miscellaneous anticonvulsants such as carbamazepine, topiramate, valproic acid, and zonisamide.

Typically, where the drug is an antidepressant, it is selected from one of the following compounds: amitriptyline, amoxapine, benmoxine, butriptyline, clomipramine, desipramine, dosulepin, doxepin, imipramine, kitanserin, lofepramine, medifoxamine, mianserin, maprotoline, mirtazapine, nortriptyline, protriptyline, trimipramine, venlafaxine, viloxazine, citalopram, cotinine, duloxetine, fluoxetine, fluvoxamine, milnacipran, nisoxetine, paroxetine, reboxetine, sertraline, tianeptine, acetaphenazine, binedaline, brofaromine, cericlamine, clovoxamine, iproniazid, isocarboxazid, moclobemide, phenyhydrazine, pheneizine, selegiline, sibutramine, tranylcypromine, ademetionine, adrafinil, amesergide, amisuipride, amperozide, benactyzine, bupropion, caroxazone, gepirone, idazoxan, metralindole, milnacipran, minaprine, nefazodone, nomifensine, ritanserin, roxindole, S-adenosylmethionine, tofenacin, trazodone, tryptophan, and zalospirone.

Typically, where the drug is an antidiabetic agent, it is selected from one of the following compounds: pioglitazone, rosiglitazone, and troglitazone.

Typically, where the drug is an antidote, it is selected from one of the following compounds: edrophonium chloride, flumazenil, deferoxamine, nalmefene, naloxone, and naltrexone.

Typically, where the drug is an antiemetic, it is selected from one of the following compounds: alizapride, azasetron, benzquinamide, bromopride, buclizine, chlorpromazine, cinnarizine, clebopride, cyclizine, diphenhydramine, diphenidol, dolasetron, droperidol, granisetron, hyoscine, lorazepam, dronabinol, metoclopramide, metopimazine, ondansetron, perphenazine, promethazine, prochlorperazine, scopolamine, triethylperazine, trifluoperazine, triflupromazine, trimethobenzamide, tropisetron, domperidone, and palonosetron.

Typically, where the drug is an antihistamine, it is selected from one of the following compounds: astemizole, azatadine, brompheniramine, carbinoxamine, cetrizine, chlorpheniramine, cinnarizine, clemastine, cyproheptadine, dexmedetomidine, diphenhydramine, doxylamine, fexofenadine, hydroxyzine, loratidine, promethazine, pyrilamine and terfenidine.

Typically, where the drug is an anti-infective agent, it is selected from one of the following classes: antivirals such as efavirenz; AIDS adjunct agents such as dapsone; aminoglycosides such as tobramycin; antifungals such as fluconazole; antimalarial agents such as quinine; antituberculosis agents such as ethambutol; β-lactams such as cefinetazole, cefazolin, cephalexin, cefoperazone, cefoxitin, cephacetrile, cephaloglycin, cephaloridine; cephalosporins, such as cephalosporin C, cephalothin; cephamycins such as cephamycin A, cephamycin B, and cephamycin C, cephapirin, cephradine; leprostatics such as clofazimine; penicillins such as ampicillin, amoxicillin, hetacillin, carfecillin, carindacillin, carbenicillin, amylpenicillin, azidocillin, benzylpenicillin, clometocillin, cloxacillin, cyclacillin, methicillin, nafcillin, 2-pentenylpenicillin, penicillin N, penicillin O, penicillin S, penicillin V, dicloxacillin; diphenicillin; heptylpenicillin; and metampicillin; quinolones such as ciprofloxacin, clinafloxacin, difloxacin, grepafloxacin, norfloxacin, ofloxacine, temafloxacin; tetracyclines such as doxycycline and oxytetracycline; miscellaneous anti-infectives such as linezolide, trimethoprim and sulfamethoxazole.

Typically, where the drug is an anti-neoplastic agent, it is selected from one of the following compounds: droloxifene, tamoxifen, and toremifene.

Typically, where the drug is an antiparkisonian drug, it is selected from one of the following compounds: amantadine, baclofen, biperiden, benztropine, orphenadrine, procyclidine, trihexyphenidyl, levodopa, carbidopa, andropinirole, apomorphine, benserazide, bromocriptine, budipine, cabergoline, eliprodil, eptastigmine, ergoline, galanthamine, lazabemide, lisuride, mazindol, memantine, mofegiline, pergolide, piribedil, pramipexole, propentofylline, rasagiline, remacemide, ropinerole, selegiline, spheramine, terguride, entacapone, and tolcapone.

Typically, where the drug is an antirheumatic agent, it is selected from one of the following compounds: diclofenac, hydroxychloroquine and methotrexate.

Typically, where the drug is an antipsychotic, it is selected from one of the following compounds: acetophenazine, alizapride, amisulpride, amoxapine, amperozide, aripiprazole, benperidol, benzquinamide, bromperidol, buramate, butaclamol, butaperazine, carphenazine, carpipramine, chlorpromazine, chlorprothixene, clocapramine, clomacran, clopenthixol, clospirazine, clothiapine, clozapine, cyamemazine, droperidol, flupenthixol, fluphenazine, fluspirilene, haloperidol, loxapine, melperone, mesoridazine, metofenazate, molindrone, olanzapine, penfluridol, pericyazine, perphenazine, pimozide, pipamerone, piperacetazine, pipotiazine, prochlorperazine, promazine, quetiapine, remoxipride, risperidone, sertindole, spiperone, sulpiride, thioridazine, thiothixene, trifluperidol, triflupromazine, trifluoperazine, ziprasidone, zotepine, and zuclopenthixol.

Typically, where the drug is an anxiolytic, it is selected from one of the following compounds: alprazolam, bromazepam, oxazepam, buspirone, hydroxyzine, mecloqualone, medetomidine, metomidate, adinazolam, chlordiazepoxide, clobenzepam, flurazepam, lorazepam, loprazolam, midazolam, alpidem, alseroxlon, amphenidone, azacyclonol, bromisovalum, captodiamine, capuride, carbcloral, carbromal, chloral betaine, enciprazine, flesinoxan, ipsapiraone, lesopitron, loxapine, methaqualone, methprylon, propanolol, tandospirone, trazadone, zopiclone, and zolpidem.

Typically, where the drug is an appetite stimulant, it is dronabinol.

Typically, where the drug is an appetite suppressant, it is selected from one of the following compounds: fenfluramine, phentermine and sibutramine.

Typically, where the drug is a blood modifier, it is selected from one of the following compounds: cilostazol and dipyridamol.

Typically, where the drug is a cardiovascular agent, it is selected from one of the following compounds: benazepril, captopril, enalapril, quinapril, ramipril, doxazosin, prazosin, clonidine, labetolol, candesartan, irbesartan, losartan, telmisartan, valsartan, disopyramide, flecanide, mexiletine, procainamide, propafenone, quinidine, tocainide, amiodarone, dofetilide, ibutilide, adenosine, gemfibrozil, lovastatin, acebutalol, atenolol, bisoprolol, esmolol, metoprolol, nadolol, pindolol, propranolol, sotalol, diltiazem, nifedipine, verapamil, spironolactone, bumetanide, ethacrynic acid, furosemide, torsemide, amiloride, triamterene, and metolazone.

Typically, where the drug is a central nervous system stimulant, it is selected from one of the following compounds: amphetamine, brucine, caffeine, dexfenfluramine, dextroamphetamine, ephedrine, fenfluramine, mazindol, methyphenidate, pemoline, phentermine, sibutramine, and modafinil.

Typically, where the drug is a drug for Alzheimer's disease management, it is selected from one of the following compounds: donepezil, galanthamine and tacrin.

Typically, where the drug is a drug for cystic fibrosis management, it is selected from one of the following compounds: tobramycin and cefadroxil.

Typically, where the drug is a diagnostic agent, it is selected from one of the following compounds: adenosine and aminohippuric acid.

Typically, where the drug is a dietary supplement, it is selected from one of the following compounds: melatonin and vitamin-E.

Typically, where the drug is a drug for erectile dysfunction, it is selected from one of the following compounds: tadalafil, sildenafil, vardenafil, apomorphine, apomorphine diacetate, phentolamine, and yohimbine.

Typically, where the drug is a gastrointestinal agent, it is selected from one of the following compounds: loperamide, atropine, hyoscyamine, famotidine, lansoprazole, omeprazole, and rebeprazole.

Typically, where the drug is a hormone, it is selected from one of the following compounds: testosterone, estradiol, and cortisone.

Typically, where the drug is a drug for the treatment of alcoholism, it is selected from one of the following compounds: naloxone, naltrexone, and disulfiram.

Typically, where the drug is a drug for the treatment of addiction it is buprenorphine.

Typically, where the drug is an immunosupressive, it is selected from one of the following compounds: mycophenolic acid, cyclosporin, azathioprine, tacrolimus, and rapamycin.

Typically, where the drug is a mast cell stabilizer, it is selected from one of the following compounds: cromolyn, pemirolast, and nedocromil.

Typically, where the drug is a drug for migraine headache, it is selected from one of the following compounds: almotriptan, alperopride, codeine, dihydroergotamine, ergotamine, eletriptan, frovatriptan, isometheptene, lidocaine, lisuride, metoclopramide, naratriptan, oxycodone, propoxyphene, rizatriptan, sumatriptan, tolfenamic acid, zolmitriptan, amitriptyline, atenolol, clonidine, cyproheptadine, diltiazem, doxepin, fluoxetine, lisinopril, methysergide, metoprolol, nadolol, nortriptyline, paroxetine, pizotifen, pizotyline, propanolol, protriptyline, sertraline, timolol, and verapamil. Prochlorperazine and other phenothiazine antipsychotics, amoxapine, and loxapine are also useful drugs for treating migraine headache within the context of the invention. See “Acute treatment of headache with phenothiazine antipsychotics,” U.S. patent application Ser. No. 10/719,763 (Publication No. US 2004-20040101481-A1).

Typically, where the drug is a motion sickness product, it is selected from one of the following compounds: diphenhydramine, promethazine, and scopolamine.

Typically, where the drug is a drug for multiple sclerosis management, it is selected from one of the following compounds: bencyclane, methylprednisolone, mitoxantrone, and prednisolone.

Typically, where the drug is a muscle relaxant, it is selected from one of the following compounds: baclofen, chlorzoxazone, cyclobenzaprine, methocarbamol, orphenadrine, quinine, and tizanidine.

Typically, where the drug is a nonsteroidal anti-inflammatory, it is selected from one of the following compounds: aceclofenac, acetaminophen, alminoprofen, amfenac, aminopropylon, amixetrine, aspirin, benoxaprofen, bromfenac, bufexamac, carprofen, celecoxib, choline, salicylate, cinchophen, cinmetacin, clopriac, clometacin, diclofenac, diflunisal, etodolac, fenoprofen, flurbiprofen, ibuprofen, indomethacin, indoprofen, ketoprofen, ketorolac, mazipredone, meclofenamate, nabumetone, naproxen, parecoxib, piroxicam, pirprofen, rofecoxib, sulindac, tolfenamate, tolmetin, and valdecoxib.

Typically, where the drug is an opioid, it is selected from one of the following compounds: alfentanil, allylprodine, alphaprodine, anileridine, benzylmorphine, bezitramide, buprenorphine, butorphanol, carbiphene, cipramadol, clonitazene, codeine, dextromoramide, dextropropoxyphene, diamorphine, dihydrocodeine, diphenoxylate, dipipanone, fentanyl, hydromorphone, L-alpha acetyl methadol, lofentanil, levorphanol, meperidine, methadone, meptazinol, metopon, morphine, nalbuphine, nalorphine, oxycodone, papaveretum, pethidine, pentazocine, phenazocine, remifentanil, sufentanil, and tramadol.

Typically, where the drug is another analgesic it is selected from one of the following compounds: apazone, benzpiperylon, benzydramine, caffeine, clonixin, ethoheptazine, flupirtine, nefopam, orphenadrine, propacetamol, and propoxyphene.

Typically, where the drug is an opthalmic preparation, it is selected from one of the following compounds: ketotifen and betaxolol.

Typically, where the drug is an osteoporosis preparation, it is selected from one of the following compounds: alendronate, estradiol, estropitate, risedronate and raloxifene.

Typically, where the drug is a prostaglandin, it is selected from one of the following compounds: epoprostanol, dinoprostone, misoprostol, and alprostadil.

Typically, where the drug is a respiratory agent, it is selected from one of the following compounds: albuterol, ephedrine, epinephrine, fomoterol, metaproterenol, terbutaline, budesonide, ciclesonide, dexamethasone, flunisolide, fluticasone propionate, triamcinolone acetonide, ipratropium bromide, pseudoephedrine, theophylline, montelukast, and zafirlukast.

Typically, where the drug is a sedative and hypnotic, it is selected from one of the following compounds: alprazolam, butalbital, chlordiazepoxide, diazepam, estazolam, flunitrazepam, flurazepam, lorazepam, midazolam, temazepam, triazolam, zaleplon, zolpidem, and zopiclone.

Typically, where the drug is a skin and mucous membrane agent, it is selected from one of the following compounds: isotretinoin, bergapten and methoxsalen.

Typically, where the drug is a smoking cessation aid, it is selected from one of the following compounds: nicotine and varenicline.

Typically, where the drug is a Tourette's syndrome agent, it is pimozide.

Typically, where the drug is a urinary tract agent, it is selected from one of the following compounds: tolteridine, darifenicin, propantheline bromide, and oxybutynin.

Typically, where the drug is a vertigo agent, it is selected from one of the following compounds: betahistine and meclizine.

Of course, drugs listed under a particular indication or class may also find utility in other indications, with alterantive uses of some of the above compounds well known to those skilled in the art.

In one embodiment of the invention, the drug active agent has a lipid relative to water solubility, as measured by the log of their octanol water partition coefficient ranging from 2 to 6, but will typically be from 3 to 5. Exemplary compounds are prochlorperazine, trifluoperazine, alprazolam, midazolam, loxapine, olanzapine, buprenorphine, sufentanyl, remifentanyl, and fentanyl.

In another embodiment of the invention, the drug active agent has certain biological and/or pharmacological properties. In particular, the drug active agents do not include drugs solely related to recreational purposes. For example, in one aspect, the drug active agents do not serve as agonists of nicotinic or cannabinoid receptors, are not vasoconstrictors, and are not bronchoconstrictors. In one aspect, the drug active agents block dopamine receptors or serotonin receptors, or serves as agonists or partial agonists of opioid or dopamine receptors, or enhance neurotransmission through GABA receptors.

Salt forms of various drug active agents that can be used in the invention are either commercially available or are obtained from the corresponding free base using well known methods in the art. A variety of pharmaceutically acceptable salts are suitable for aerosolization. Such salts include, without limitation, the following: hydrochloric acid, hydrobromic acid, acetic acid, maleic acid, formic acid, and fumaric acid salts.

Pharmaceutically acceptable excipients may be volatile or nonvolatile. Volatile excipients, when heated, are concurrently volatilized, aerosolized and inhaled with the drug active agent. Classes of such excipients are known in the art and include, without limitation, gaseous, supercritical fluid, liquid and solid solvents. The following is a list of exemplary carriers within the classes: water; terpenes, such as menthol; alcohols, such as ethanol, propylene glycol, glycerol and other similar alcohols; dimethylformamide; dimethylacetamide; wax; supercritical carbon dioxide; dry ice; and mixtures thereof.

In another embodiment, the aerosols of the invention also have certain properties. In particular, the aerosols have a mass median aerodynamic diameter (MMAD) of between about 0.8 μm and 5 μm, typically about 1 μm to 4 μm, and most commonly about 1 μm to 3 μm. The aerosol typically has a geometric standard deviation around the MMAD of less than 4, 3, 2.5, 2.2, or 2. The aerosol is typically in liquid form. Alternatively, the aerosol, if in solid form, is in amorphous rather than crystalline form. Typically, the aerosol particles comprise about 20%, 40%, 60%, 80%, 90%, 95%, or 97% drug active agent, as opposed to additive or solvent. The fraction of drug active agent and/or drug in aerosol particles may be determined by collecting the aerosol particles (e.g., in a cold trap or filter) and weighing the trapped particles, and thereafter extracting the trap to determine the quantity of drug active agent in the trap by an analytical means (e.g., liquid chromatography with ultraviolet light detection), and dividing the amount of drug active agent in the trap as measured by the analytical means by the weight of collected particles. In one aspect, the aerosol is a thermally generated aerosol comprising vaporized drug active agent condensed into particles typically comprising less than 20%, 10% 5%, or 3% thermal decomposition products.

To determine the percent fraction of drug degradation products, the aerosol is typically collected in a trap, such as a filter, glass wool, an impinger, a solvent trap, or a cold trap, with collection in a filter being the common technique used. The trap is then extracted with a solvent, e.g. acetonitrile, and the extract subjected to analysis by any of a variety of analytical methods known in the art, with gas and liquid chromatography methods typically being used, and high performance liquid chromatography (HPLC) particularly useful. The gas or liquid chromatography method includes a detector system such as a mass spectrometry detector or ultraviolet absorption detector. Ideally, the detector system allows determination of the quantity of the components of the drug composition and drug degradation product by weight. This is achieved in practice by measuring the signal obtained upon analysis of one or more known components of the drug composition or drug degradation product (standards) and comparing the signal obtained upon analysis of the aerosol to that obtained upon analysis of the standard(s), an approach well known in the art.

In many cases, the structure of a drug degradation product may not be known or a standard of the drug degradation product may not be available. In such cases, it is acceptable to calculate the weight fraction of the drug degradation product by assuming that the drug degradation product has an identical response coefficient (e.g., for ultraviolet absorption detection, identical extinction coefficient) to the drug component or components in the drug composition. When conducting such analysis, for purposes of practicality, drug degradation products present at less than a very small fraction of the drug, e.g., less than 0.2% or 0.1% or 0.03% of the drug, are generally excluded from analysis. Because of the frequent necessity to assume an identical response coefficient between drug and drug degradation product in calculating a weight percentage of drug degradation product, it is preferred to use an analytical approach in which such an assumption has a high probability of validity. In this respect, high performance liquid chromatography with detection by absorption of ultraviolet light at 225 nm is typically used. UV absorption at other than 225 nm, most commonly 250 nm, is used for detection of compounds in cases where the compound absorbs substantially more strongly at 250 nm or for other reasons one skilled in the art would consider detection at 250 nm the most appropriate means of estimating purity by weight using HPLC analysis. In certain cases where analysis of the drug by UV is not viable, other analytical tools such as GC/MS or LC/MS may be used to determine purity.

Using the methods of the invention, an inhalation (either single dose or multi-dose) device can be made such that the device releases one or more doses of aerosol that, when inhaled by a mammal, result in a spike index of between about 1.5 and 10.

The drug composition can be formulated for delivery in any number devices. One of skill in the art will be capable of formulating the proper dosage based upon the type of delivery device and system used the type of drug being delivered, the percentage of drug degradation during use and storage, and the like. A few exemplary delivery devices and systems are described herein.

Any suitable method can be used to form the aerosols according to the invention. For example, in one aspect the method involves heating a drug active agent to form a vapor, followed by cooling of the vapor such that it condenses to provide an aerosol (e.g., a condensation aerosol). The composition comprising the drug active agent is heated in one of four forms: as pure drug active agent; as a mixture of drug active agents and a pharmaceutically acceptable excipient; as a salt form of the pure drug active agent; and, as a mixture of drug active agent salt and a pharmaceutically acceptable excipient.

In one embodiment, a drug active agent is coated on a thermally conductive solid support. Typically, the drug composition film coated on the solid support has a thickness of between about 0.05-20 μm, and typically a thickness between 0.1-15 μm. More typically, the thickness is between about 0.2-10 μm; even more typically, the thickness is between about 0.5-10 μm, and most typically, the thickness is between about 0.5-5 μm. The desirable film thickness for any given drug composition is typically determined by an iterative process in which the desired yield and purity of the condensation aerosol composition are selected or known.

For example, if the purity of the particles is less than that which is desired, or if the percent yield is less than that which is desired, the thickness of the drug film is adjusted to a thickness different from the initial film thickness. The purity and yield are then determined at the adjusted film thickness, and this process is repeated until the desired purity and yield are achieved. After selection of an appropriate film thickness, the area of solid support required to provide a therapeutically effective dose is determined.

Generally, the film thickness for a given drug composition is such that drug-aerosol particles, formed by vaporizing the drug composition by heating the solid support and entraining the vapor in a gas stream, have (i) 10% by weight or less drug-degradation product, more preferably 5% by weight or less, most preferably 2.5% by weight or less and (ii) at least 50% of the total amount of drug composition contained in the film.

Solid supports on which the composition is heated can be any number of a variety of shapes. Examples of such shapes include, without limitation, spheres, cylinders, rectangular structures (including substantially planar structures) and the like. In one aspect, the solid support provides a large surface to volume ratio and a large surface to mass ratio.

A solid support of one shape can also be transformed into another shape with different properties. For example, a flat sheet of 0.25 mm thickness has a surface to volume ratio of approximately 8,000 per meter. Rolling the sheet into a hollow cylinder of 1 cm diameter produces a support that retains the high surface to mass ratio of the original sheet but has a lower surface to volume ratio (about 400 per meter).

A number of different materials can be used to construct a solid support. Classes of such materials include, without limitation, metals, inorganic materials, carbonaceous materials and polymers. The following are examples of the material classes: aluminum, silver, gold, stainless steel, copper and tungsten; silica, glass, silicon and alumina; graphite, porous carbons, carbon yarns and carbon felts; polytetrafluoroethylene and polyethylene glycol. Combinations of materials and coated variants of materials can be used as well.

Where aluminum is used as a solid support, aluminum foil is a suitable material. Examples of silica, alumina and silicon based materials include amphorous silica S-5631 (Sigma, St. Louis, Mo.), BCR171 (from Aldrich, St. Louis, Mo.) and a silicon wafer as used in the semiconductor industry. Carbon yarns and felts are available from American Kynol, Inc., New York, N.Y. Chromatography resins such as octadecycl silane chemically bonded to porous silica are exemplary coated variants of silica.

The heating of the compositions can be performed using any suitable method. Examples of methods by which heat can be generated include the following: passage of current through an electrical resistance element; absorption of electromagnetic radiation, such as microwave or laser light; and, exothermic chemical reactions, such as exothermic salvation, hydration of pyrophoric materials and oxidation of combustible materials.

Aerosols compositions comprising a drug active agent are delivered to a mammal using an inhalation device. Where the aerosol is a condensation aerosol, the device has at least three elements: an element for heating the composition to form a vapor; an element allowing the vapor to cool, thereby providing a condensation aerosol; and, an element permitting the mammal to inhale the aerosol. Various suitable heating methods are described above. The element that allows cooling is, in it simplest form, an inert passageway located between a heating chamber and an inhalation port. The element permitting inhalation is an aerosol exit portal that forms a connection between the cooling element and the mammal's respiratory system.

The dosage of a composition using the inhalation device described above can be regulated by providing or layering a proper therapeutic dose on the solid substrate.

An automatic aerosol administration system was used to administer the drug aerosols in vivo dog experiments described in Examples 1 and 2. The aerosol administration system was designed to produce a breathing maneuver for optimizing deep lung inhalation delivery, i.e., a full exhalation, followed by a deep inhalation, followed by a breath hold of several seconds. The breathing of the experimental animal and the timing of the drug aerosol during a breath cycle were controlled by the aerosol administration system. The breath hold time and exhalation also were controlled by the aerosol administration system.

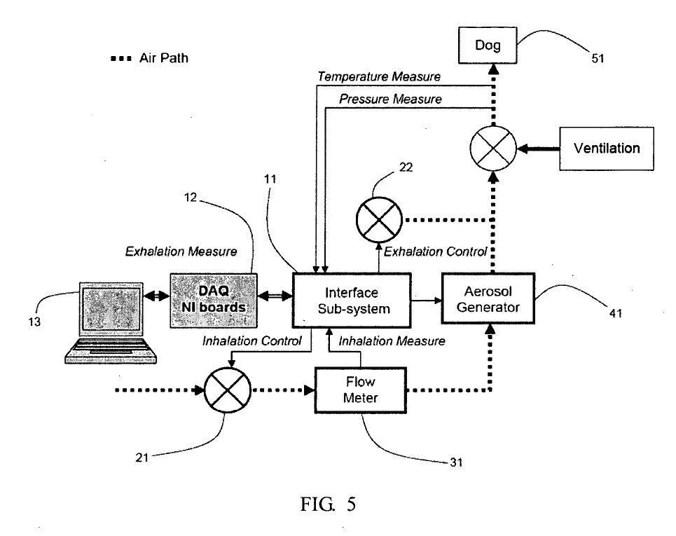

Referring to FIG. 5, the aerosol administration system is controlled through interface subsystem 11, data acquisition board 12, and laptop computer 13. On starting the aerosol administration system, computer 13 opens inhalation valve 21 starting a flow through condensation aerosol generator 41 into anesthetized dog 51. Flow meter 31 (e.g., TSI model 4045 thermal mass flow meter) is the primary sensor in the aerosol administration system. From flow meter 31, computer 13 monitors the flow rate and calculates the volume of inspiration. The inhalation valve 21 remains open until a prescribed volume of air has been delivered. When the prescribed volume is reached, inhalation valve 21 is closed and a breath hold timer starts. Typically, the breath hold timer is set for 5 seconds. When the breath hold timer expires, the exhalation valve 22 opens and the dog exhales through a filter.

When a dose of the drug active agent is to be delivered, the condensation aerosol generator 41 is triggered to produce a drug aerosol. Preferably, the condensation aerosol generator 41 is triggered early in an inhalation cycle so that most of the drug aerosol is carried in the first third of the volume of air delivered to the dog. When the prescribed volume is reached, inhalation valve 21 is closed and a breath hold timer starts. When the breath hold timer expires, the exhalation valve 22 opens and the dog exhales through a filter and is returned to maintenance anesthesia.

The aerosol administration system device may incorporate a pressure sensor and a thermocouple to assure the safety of the experimental animal. If prescribed limits of pressure or temperature are met on either sensor, the inhalation flow is immediately stopped and the exhalation valve 22 is opened to prevent injury to the animal.

Other inhalation devices can also be used in the methods and delivery dosages of the invention. For example, such devices include dry powder inhalers (DPI's), nebulizers and pressurized metered dose inhalers. Nebulizers generate an aerosol from a liquid, some by breakup of a liquid jet and some by ultrasonic vibration of the liquid with or without a nozzle. Pressurized metered dose inhalers, or pMDIs, are an additional class of aerosol dispensing devices. Pressurized metered dose inhalers package the drug composition in a canister under pressure with a solvent and propellant mixture, usually chlorofluorocarbons (CFC's,), or hydroflouroalkanes (HFA's). Upon being dispensed a jet of the mixture is ejected through a valve and nozzle and the propellant “flashes off” leaving an aerosol of the compound.

The delivery devices, if desired, can comprise a variety of components to facilitate the delivery of aerosols. For instance, the device may include any component known in the art to control the timing of drug aerosolization relative to inhalation (e.g., breath-actuation), to provide feedback to a subject on the rate and/or volume of inhalation, to prevent excessive use (i.e., “lock-out” feature), to prevent use by unauthorized individuals, and/or to record dosing histories.

One can determine the appropriate dose of drug composition containing aerosols to treat a particular condition in humans using the methods described herein in combination with animal experiments and a dose-finding (Phase I/II) clinical trial. Such animal experiments involve measuring plasma concentrations of drug in an animal after its exposure to the aerosol and IV injections in order to determine the spike index. Mammals such as dogs or primates are typically used in such studies, since their respiratory systems are similar to that of a human. Initial dose levels for testing in humans are generally less than or equal to the dose in the mammal model that resulted in plasma drug concentrations associated with a therapeutic effect in humans and/or associated with a significant side effect in humans or animal models, with starting doses in humans generally at least 2, 5, or 10-fold less than doses that cause substantially toxicity in animal toxicology or safety pharmacology studies. Dose escalation in humans is then performed, until either an optimal therapeutic response is obtained or a dose-limiting toxicity is encountered.

Particle size distribution of a drug containing aerosol is determined using any suitable method in the art (e.g., cascade impaction). An Andersen Eight Stage Non-viable Cascade Impactor (Andersen Instruments, Smyrna, Ga.) linked to the aerosol generator or other inhalation device by a mock throat (USP throat, Andersen Instruments, Smyrna, Ga.) is one system used for cascade impaction studies.

Inhalable aerosol drug mass density is determined, for example, by delivering a drug-containing aerosol into a confined chamber via an inhalation device and measuring the amount of active drug collected in the chamber. Typically, the aerosol is drawn into the chamber by having a pressure gradient between the device and the chamber, wherein the chamber is at lower pressure than the device. The volume of the chamber should approximate the tidal volume of an inhaling subject. The amount of drug collected in the chamber is determined by extracting the chamber, conducting chromatographic analysis of the extract and comparing the results of the chromatographic analysis to those of a standard containing known amounts of drug.

Inhalable aerosol particle density is determined, for example, by delivering aerosol phase drug into a confined chamber via an inhalation device and measuring the number of particles of given size collected in the chamber. The number of particles of a given size may be directly measured based on the light-scattering properties of the particles. Alternatively, the number of particles of a given size is determined by measuring the mass of particles within the given size range and calculating the number of particles based on the mass as follows: Total number of particles=Sum (from size range 1 to size range N) of number of particles in each size range. Number of particles in a given size range=Mass in the size range/Mass of a typical particle in the size range. Mass of a typical particle in a given size range=π*D³*φ/6, where D is a typical particle diameter in the size range (generally, the mean boundary MMADs defining the size range) in microns, φ is the particle density (in g/mL) and mass is given in units of picograms (g⁻¹²).

Rate of inhalable aerosol particle formation is determined, for example, by delivering an aerosol phase drug into a confined chamber via an inhalation device. The delivery is for a set period of time (e.g., 3 sec), and the number of particles of a given size collected in the chamber is determined as outlined above. The rate of particle formation is equal to the number of 100 nm to 5 micron particles collected divided by the duration of the collection time.

Rate of aerosol formation is determined, for example, by delivering aerosol phase drug into a confined chamber via an inhalation device. The delivery is for a set period of time (e.g., 3 sec), and the mass of particulate matter collected is determined by weighing the confined chamber before and after the delivery of the particulate matter. The rate of aerosol formation is equal to the increase in mass in the chamber divided by the duration of the collection time. Alternatively, where a change in mass of the delivery device or component thereof can only occur through release of the aerosol phase particulate matter, the mass of particulate matter may be equated with the mass lost from the device or component during the delivery of the aerosol. In this case, the rate of aerosol formation is equal to the decrease in mass of the device or component during the delivery event divided by the duration of the delivery event.

Rate of drug aerosol formation is determined, for example, by delivering a drug containing aerosol into a confined chamber via an inhalation device over a set period of time (e.g., 3 sec). Where the aerosol is pure drug active agent, the amount of drug active agent collected in the chamber is measured as described above. The rate of drug aerosol formation is equal to the amount of drug collected in the chamber divided by the duration of the collection time. Where the drug containing aerosol comprises a pharmaceutically acceptable excipient, multiplying the rate of aerosol formation by the percentage of drug in the aerosol provides the rate of drug aerosol formation.

### EXAMPLES

### Example 1

### Pharmacokinetics of an Aerosolized Prochlorperazine in Dog

The objective of this study was to determine the concentration of prochlorperazine in dog plasma processed from whole blood taken from both the left ventricle and venous circulation immediately following administration of prochlorperazine aerosol to the animal by way of the thermal aerosol generation process described herein, during a single deep inhalation. The plasma levels thus obtained were compared to those obtained by two methods of intravenous administration (bolus and infusion) of prochlorperazine solution for injection.

This study consisted of four young adult, female, mongrel dogs. The animals were treated with the test article, prochlorperazine (PCZ), in three separate surgical/dosing sessions. In sessions 1 and 3, the test article was administered intravenously (IV), either via a 120-second infusion (Session 1) or as a five-second bolus (Session 3). In session 2, the test article was administered using a thermal aerosol generation process, during a single deep inhalation. Following treatment in each surgical/dosing session, blood samples were collected from the left ventricle and from a peripheral vein for bioanalysis. Left ventricular and venous plasma samples were obtained prior to drug administration. In addition, left ventricular plasma samples were obtained every five seconds during the first 30 seconds following drug administration, as well as frequently thereafter, with the last left ventricular sample collected 10 minutes following drug administration. Venous plasma samples were obtained from 15 seconds to 24 hours following drug administration. Clinical observations and body weights were recorded for all animals at specified time points.

There were no adverse effects noted by clinical observations that could be attributed to the administration of PCZ. Body weights were maintained in all animals between treatment sessions.

Plasma concentrations of PCZ rose during the two-minute IV infusion to reach a mean Cₘₐₓ of 1365±396 ng/mL in left ventricular plasma and 442±270 ng/mL in venous plasma at or near the end of the infusion. In contrast, plasma concentrations rose very rapidly after both the aerosol and IV bolus treatments, reaching maximum plasma levels within approximately 20 to 30 seconds after dosing. Maximum left ventricular plasma concentrations were similar after aerosol (3262±975 ng/mL) and IV bolus (3482±767 ng/mL) administration. Plasma concentration vs. time profiles in both left ventricular and venous plasma were nearly identical for aerosol and IV bolus treatments. For the first 60 seconds after administration, left ventricular PCZ levels for the aerosol and IV bolus treatments exceeded those for the two-minute IV infusion treatment, but at later time points concentrations were similar for all three treatments. Left ventricular plasma AUCs during the 10-minute sampling period were similar for all three treatments (88 to 98 ng/hr/mL).

Overall, venous pharmacokinetics were similar between the three groups, with mean clearances ranging from 18.9 to 24.8 mL/min/kg, mean half-lives ranging from 1.27 to 1.75 hours, and mean volumes of distribution between 2.3 and 3.8 L/kg. The PCZ delivered via aerosol had a bioavailability of 82%±13% compared to the IV bolus and 109%±19% compared to the two-minute IV infusion.

### Frequency and Duration of Treatment Administration

The animals were treated in three separate surgical/dosing sessions. For each session, the animals were appropriately anesthetized prior to administration of the test article. Each surgical/dosing session was separated by a washout period of approximately 48 hours from the time of the previous treatment administration.

Session 1: While the animals were anesthetized, the test article (1.4 mL of 5 mg/mL prochloperazine edisylate injection) was administered as a 120-second intravenous (IV) manual infusion via a saphenous vein. Blood sampling commenced at the time of initiation of infusion (t=0).

Session 2: While the animals were anesthetized, an aerosol generation and administration system was connected in-line to an endotracheal tube to allow for delivery of the aerosol test article (prochlorperazine coated on a chemical, single dose heat package). The aerosol resulting from actuation of the prochlorperazine by the aerosol generation and administration system was administered during a single deep inhalation. Blood sampling commenced at the time of initiation of the deep inhalation (t=0).

Session 3: While the animals were anesthetized, the test article (1.4 mL of 5 mg/mL prochloperazine edisylate injection) was administered as a five-second IV bolus via a saphenous vein. Blood sampling commenced at the time of initiation of the bolus (t=0).

### Anesthesia

Each animal was premedicated with atropine sulfate (0.02 mg/kg, intramuscularly) and acepromazine (0.2 mg/kg, IM, to a maximum dose of 3 mg) prior to induction of anesthesia. At least 10 minutes later, the animal was anesthetized with Propofol(4-8 mg/kg, intravenously). The animal was then intubated and maintained in anesthesia with isoflurane inhalant anesthetic, delivered through a volume-regulated ventilator. An intravenous catheter was placed in a peripheral vessel for administration of lactated Ringer's solution during the procedure at a rate of approximately 5 mL/kg/hr.

### Surgical Procedure

A midline incision was made in the neck and one of the carotid arteries was exposed. The artery was mobilized a distance of about 5 cm and two Vessel-Loops® were placed around it, proximally and distally. The loops were both tightened to temporarily occlude blood flow, and a small arteriotomy was made to allow the introduction of a specially designed 7 Fr CBAS catheter with a volume of 1.0 mL throughout its length. The distal tip of this catheter was introduced into the left ventricle (or placed as close as possible to this location) via fluoroscopic guidance. The proximal end of the catheter was capped with a three-way stopcock and the catheter was filled with an isotonic solution. The jugular vein was also exposed and isolated in a similar manner, and an appropriately sized catheter was passed just into the vessel to facilitate venous blood collection.

Treatment was then administered, and the necessary blood collections were performed from the left ventricular and venous catheters, as described below.

One mL of blood was aspirated into the left ventricular catheter. This sample was expelled into an appropriately labeled blood tube through a double acting check valve following aspiration of the next time point. This sampling procedure was used for the initial time points (where there were only seconds in between), until there was at least 30 seconds between time points. The remaining left ventricular blood samples were taken conventionally, in that the catheter was flushed with an isotonic solution before the collection of the next sample. Venous blood samples were collected conventionally.

When the necessary blood collection was completed (after the 10-minute time point), the ventricular catheter was removed, the Vessel-Loops® were tightened, and the arteriotomy was repaired with a simple continuous pattern of 6-0 Prolene® suture material, so as to allow for its use during subsequent surgical/dosing sessions. A similar procedure was used to repair the jugular vein following the 30- or 60-minute time point and the remaining venous samples were obtained conventionally by percutaneous stick from a cephalic vein.

The neck incision was then closed in layers, and the skin was closed with an absorbable suture placed in a subcuticular pattern.

TABLE 1
Blood Sample Collection Schedule

| Session | Site of | Time Points |
|---|---|---|
| Number | Collection | (Time Post t = 0)^{a} |
| 1 | Left Ventricle | Start of treatment, and then 5, 10, 15, 20, 25, 30, 40, 50, 60, |
| | | 75, 90, 120, 150, and 180 seconds, and 5 and 10 |
| | | minutes post t = 0 |
| | Venous | Prior to treatment and then 15, 30, 60, 120, and 180 |
| | | seconds, 5, 10, 20, 30, 60, 120, and 240 minutes, and 8, |
| | | 12, 16, and 24 hours post t = 0 |
| 2 | Left Ventricle | Start of treatment, and then 5, 10, 15, 20, 25, 30, 40, 50, |
| | | 60, 75, 90, 120, 150, and 180 seconds, and 5 and 10 |
| | | minutes post t = 0 |
| | Venous | Prior to treatment and then 15, 30, 60, 120, and 180 |
| | | seconds, 5, 10, 20, 30, 60, 120, and 240 minutes, and 8, |
| | | 12, 16, and 24 hours post t = 0 |
| 3 | Left Ventricle | Start of treatment, and then 5, 10, 15, 20, 25, 30, 40, 50, |
| | | 60, 75, 90, 120, 150, and 180 seconds, and 5 and 10 |
| | | minutes post t = 0 |
| | Venous | Prior to treatment and then 15, 30, 60, 120, and 180 |
| | | seconds, 5, 10, 20, 30, 60, 120, and 240 minutes, and 8, |
| | | 12, 16, and 24 hours post t = 0 |

^{a}A two-second deviation was allowed on all time points out to and including the 60-second time point. Following this period, deviations for sampling times were determined per Test Facility SOP (±5% of each time point). Collection times were recorded in hour/minute/second format for at least up to and including the 30-minute time point, following which collection time points were recorded in hour/minute format.

### Bioanalytical Samples

Whole blood samples were centrifuged. The plasma was extracted and placed in a −70° C. freezer within 90 minutes from the time of collection. All plasma samples were stored until analyzed. Concentrations of PCZ in plasma samples were measured using a validated LC-MS/MS method with a limit of quantitation of 2.0 ng/mL. Plasma samples were mixed with internal standard (2H3-prochlorperazine) and sodium bicarbonate then purified using solid phase extraction (Waters Oasis HLB). Extracts were separated by gradient HPLC on a Phenomenex Synergi Hydro-RP, 4 micron column and subjected to tandem mass spectrometry (MDS Sciex API 3000) with electrospray ionization in positive ion mode and mass ratio monitoring (MRM) detection. Drug concentrations were calculated by comparing PCZ/internal standard ratios to a standard curve (2.0 to 400 ng/mL PCZ) prepared in dog plasma.

### Dose Formulation Analysis

Aerosol samples were captured from the aerosol administration system before and after dose administration. These samples were captured in either a single filter to measure Emitted Dose (ED) or in an Anderson Cascade Impactor (ACI) to measure particle size distribution. The analyses confirmed that the aerosol administration system, when loaded with the test article (prochlorperazine) generated the appropriate aerosol emitted from the dog's endotracheal tube, (i.e., a prochlorperazine emitted dose of approximately 7; (e.g., between 6.0 and 7.3 mg in all test samples' mean pretreatment 6.71 mg and post treatment 6.70 mg; with an aerosol mass median aerodynamic diameter of approximately 2.2 micrometers; between 2.0 and 2.3 micrometers in all test samples' mean pretreatment 2.1 micrometers and post treatment 2.3 micrometers and geometric standard deviation of 1.8).

TABLE 2
Data Summary for Stability of Aerosol

| Test | Before Dosing | | After Dosing | |
|---|---|---|---|---|
| Emitted Dose | 1. 7.14 mg | | 1. 6.86 mg | |
| | 2. 7.00 mg | | 2. 6.01 mg | |
| | 3. 6.01 mg | | 3. 7.24 mg | |
| | Average = 6.71 mg | | Average = 6.70 mg | |
| | SD = 0.62 mg | | SD = 0.63 mg | |
| Aerosol Particle | | | | |
| Size Distribution | MMAD, μm | GSD | MMAD, μm | GSD |
| Coated Drug | 2.2 | 1.8 | 2.3 | 1.8 |
| Identity and | 2.2 | 1.8 | 2.3 | 1.8 |
| Impurities | 2.0 | 1.8 | 2.2 | 1.8 |
| | Ave = 2.1 | Ave = 1.8 | Ave = 2.3 | Ave = 1.8 |

| | | |
|---|---|---|
| Coated Drug | PCZ sulfoxide = 0.03% | PCZ sulfoxide = 0.03% |
| Identity and | Perazine = 0.11% | Perazine = 0.11% |
| Impurities | 2-Chlorophenothiazine = | 2-Chlorophenothiazine = |
| | 0.95% | 1.03% |
| | RRT 0.97 = 0.14% | RRT 0.97 = 0.15% |
| | RRT 1.05 = 0.24% | RRT 1.05 = 0.23% |
| | RRT 1.35 = 0.44% | RRT 1.35 = 0.46% |
| | RRT 1.59 = 0.12% | RRT 1.59 = 0.11% |
| | RRT 1.63 = 0.24% | RRT 1.63 = 0.23% |
| | Total Impurities = 2.47% | Total Impurities = 2.60% |

MMAD = Mass Medium Aerodynamic Diameter
GSD = Geometric Standard Deviation
RRT = Relative Retention Time

### Pharmacokinetic Analysis Procedures

The plasma concentration vs. time data were analyzed using non-compartmental pharmacokinetic methods. Pharmacokinetic parameters were determined from each animal's left ventricular and venous plasma concentration data after each treatment.

The maximum observed plasma concentration (Cₘₐₓ) and the time at which Cₘₐₓ occurred (Tₘₐₓ) were determined by inspection. Areas under the plasma concentration vs. time curve was determined by linear trapezoidal integration, with the assumption that concentration at time zero was zero. Left ventricular plasma AUCs were determined from time zero to the last time point at 10 minutes (AUC₀₋₁₀ₘᵢₙ). Venous plasma AUCs were determined from time zero to 10 minutes (AUC₀₋₁₀ₘᵢₙ), from time zero to the last measurable time point, Tₗₐₛₜ (AUC₀₋ₗₐₛₜ) and from zero to infinity (AUC_{inf}). Values of AUC_{inf} were determined as: AUC_{inf}=AUC₀₋ₗₐₛₜ+(Cₗₐₛₜ*half-life)/ln2, where Cₗₐₛₜ was the plasma concentration at time Tₗₐₛₜ. Half-lives were determined by fitting the terminal log-linear portion (at least three points) of the plasma concentration vs. time curve using a non-linear, least-squares minimization algorithm (RSTRIP, MicroMath, version 5.0). Other pharmacokinetic calculations were performed using Microsoft® Excel 2000, version 9.0. Clearance was determined as Dose/AUC_{inf} and Volume of Distribution as V_{d}=CL/k where k was 0.693/half-life. Bioavailability was determined as the ratio of AUC_{inf} after aerosol administration to the AUC_{inf} after intravenous administration.

### Pharmacokinetic

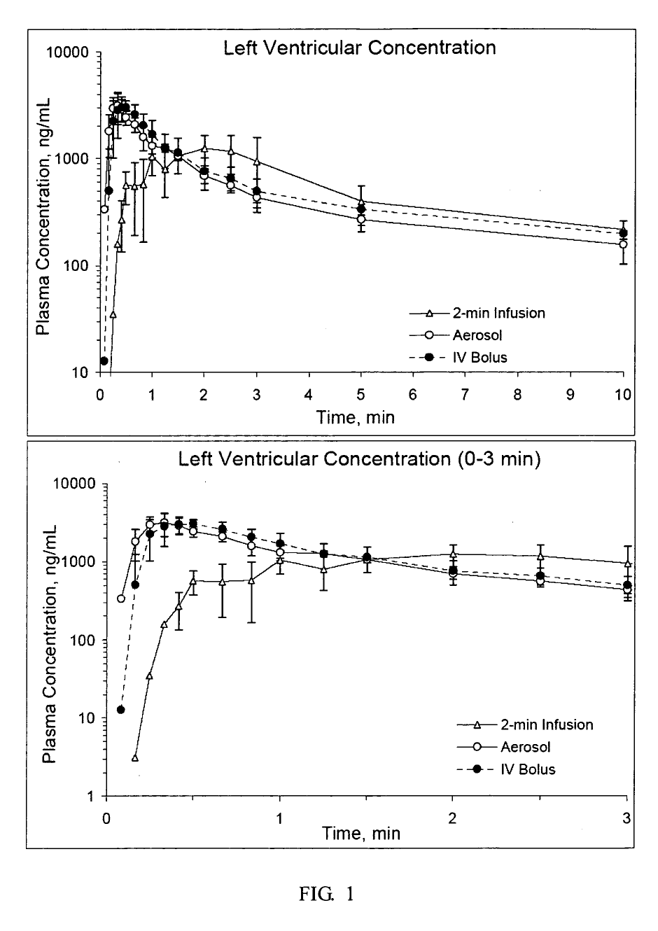

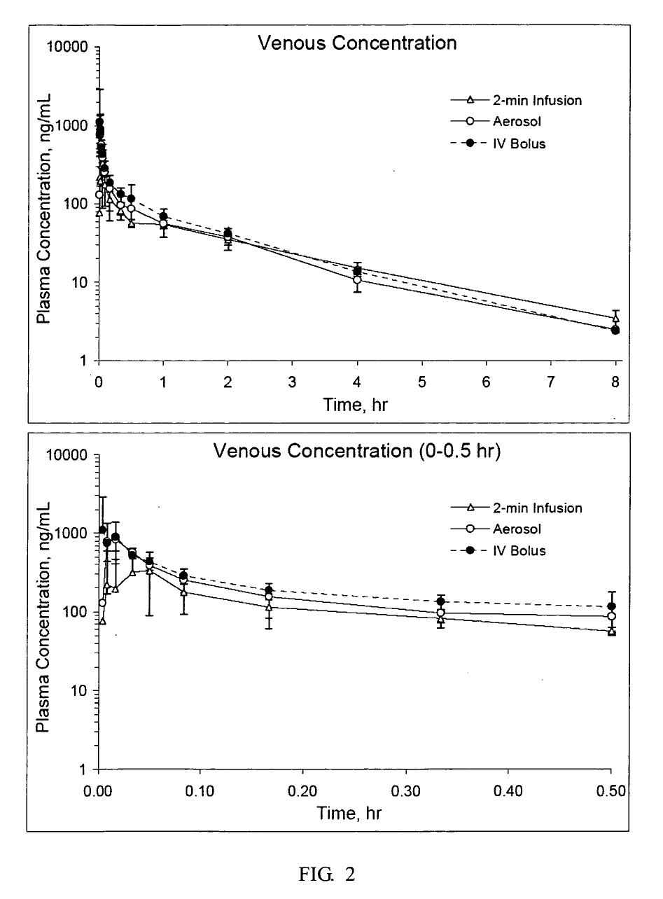

In this comparative pharmacokinetic study, dogs received 7 mg of PCZ as a 2-minute IV infusion, a single deep inhalation of aerosol, and an IV bolus in three consecutive treatment sessions. Left ventricular and venous pharmacokinetic parameters of PCZ were determined and are summarized in Table 3. Concentrations of PCZ in left ventricular plasma are plotted in FIG. 1. Venous concentrations of PCZ are plotted in FIG. 2.

After administration of a 2-minute IV infusion of PCZ (Session 1), PCZ concentrations in left ventricular and venous plasma peaked at or near the end of the infusion (Tₘₐₓ, 1.25 to 3 min). Mean maximum concentrations were approximately 3-fold higher in left ventricular plasma (1365±396 ng/mL) than in venous plasma (442±270 ng/mL). Venous plasma concentrations fell with a terminal half-life of 1.75±0.16 hr, to undetectable levels (<2 ng/mL) at time points after 8 hr. The clearance (24.8 mL/min·kg) and volume of distribution (3.8 L/kg) were similar to those of PCZ in humans, confirming that PCZ is extensively eliminated and widely distributed in tissues in both species.

After administration of a single-breath thermal aerosol of PCZ (Session 2), PCZ concentrations rose very rapidly in left ventricular and venous plasma (FIGS. 1-2).

Maximum concentrations in left ventricular plasma (3262±975 ng/mL) were reached at 0.33±0.07 minutes (20 seconds) after the inhalation. Maximum concentrations in venous plasma (886±268 ng/mL) were reached at 1.0±0.7 minutes. After the IV bolus administration (Session 3), PCZ concentrations at very early time points were lower than those after the aerosol exposure, but nevertheless did rise rapidly to values similar to those achieved after aerosol exposure, with left ventricular concentrations reaching 3482±767 ng/mL at 0.50±0.14 minutes (30 seconds) after the injection, and venous concentrations reaching 1301±1265 at 1.1±0.7 minutes. The concentration vs. time profiles in left ventricular plasma were nearly identical for aerosol and IV bolus administration over the 10 minute duration of left ventricular sampling (FIG. 1). For approximately 60 seconds after administration, left ventricular PCZ levels for the aerosol and IV bolus treatments exceeded those observed for the 2-minute IV infusion treatment (FIG. 1). At later time points, left ventricular concentrations were similar for all three treatments. Although peak PCZ concentrations in left ventricular plasma were approximately 2.5-fold higher for the aerosol and IV bolus treatments, the acute left ventricular exposure (AUC₀₋₁₀ₘᵢₙ) was similar for all three treatments (Table 3). Left ventricular concentrations were higher than venous concentrations during the first few minutes after each treatment, but this concentration difference had nearly vanished by the end of the 10-minute left ventricular sampling period, indicating that absorption was rapid after all three treatments. Venous concentrations at time points greater than 5 minutes were of similar magnitude, and declined with a similar half-life for all three treatments (FIG. 2). As a result the total venous PCZ exposure (AUC_{inf}), half-life, clearance (CL) and volume of distribution (Vd) were similar for the three routes of administration. The bioavailability of the PCZ aerosol, based on venous AUC_{inf}, was 82±13% when compared to the IV bolus and 108.7±19% when compared to the 2-minute infusion at the same dose. In comparison, the oral bioavailability of PCZ is reported to be only 12.5% in humans. The (ventricular) spike index for inhalation of prochlorperazine was 3261 ng/mL divided by 1365 ng/mL=2.4.

TABLE 3
Summary of PCZ Pharmacokinetic Parameters by Treatment

| | | | | |
|---|---|---|---|---|
| | | 2-min | Aerosol | IV Bolus |
| Parameter | Units | Infusion Mean ± SD | Mean ± SD | Mean ± SD |

Pharmacokinetic Parameters from Venous Plasma Concentrations

| | | | | |
|---|---|---|---|---|
| Cₘₐₓ | ng/mL | 441.8 ± 269.9 | 886.5 ± 268.3 | 1301 ± 1265 |
| Tₘₐₓ | Min | 2.5 ± 0.6 | 1.0 ± 0.7 | 1.1 ± 0.7 |
| Tₗₐₛₜ | Hr | 8.0 ± 0.0 | 6.0 ± 2.3 | 8.0 ± 0.0 |
| AUC_{0-10 min} | Ng hr/mL | 32.2 ± 21.1 | 56.5 ± 10.6 | 63.4 ± 15.7 |
| AUC_{inf} | Ng hr/mL | 229.9 ± 35.7 | 250.4 ± 58.2 | 305.1 ± 59.3 |
| CL | mL/min kg | 24.8 ± 2.9 | 23.0 ± 4.9 | 18.9 ± 3.6 |
| Half-Life | Hr | 1.75 ± 0.16 | 1.27 ± 0.29 | 1.37 ± 0.10 |
| Vd | L/kg | 3.8 ± 0.7 | 2.6 ± 1.1 | 2.3 ± 0.6 |
| F^{a} | % | NA | 82.1 ± 13.1 | NA |
| F^{b} | % | NA | 108.9 ± 19.2 | NA |

Pharmacokinetic Parameters from Left Ventricular Plasma Concentrations

| | | | | |
|---|---|---|---|---|
| Cₘₐₓ | ng/mL | 1365.3 ± 396.2 | 3261.5 ± 974.8 | 3481.8 ± 787.2 |
| Tₘₐₓ | min | 1.9 ± 0.8 | 0.33 ± 0.07 | 0.50 ± 0.14 |
| AUC_{0-10 min} | ng hr/mL | 88.5 ± 25.7 | 89.1 ± 13.7 | 98.3 ± 15.0 |

^{a}Aerosol bioavalability to IV bolus
^{b}Aerosol bioavalability to IV Infusion

### Example 2

### Pharmacokinetics of an Aerosolized Alprazolam in Dog

The objective of this study was to determine the concentration of alprazolam within the blood of the left ventricle immediately after being administered to the animal by way of the thermal aerosol generation process described herein, during a single deep inhalation. The plasma levels thus obtained were compared to those obtained by intravenous administration of the same test article. This study consisted of five young adult, female, mongrel dogs. Four animals per session were treated with the test article, alprazolam, in two separate surgical/dosing sessions. In Session 1 the test article was administered intravenously (IV) as a 5-second bolus. In Session 2, the test article was administered via the thermal aerosol generation process described herein, during a single deep inhalation. The animals were dosed at a level of 0.7 mg of alprazolam in both dosing sessions. Following treatment in each surgical/dosing session, blood samples were collected from the left ventricle and from a peripheral vein for bioanalysis by PHARMout® Laboratories. Left ventricular plasma samples were obtained at the start of dosing, and then every 5 seconds during the first 30 seconds following drug administration, as well as frequently thereafter, with the last left ventricular sample collected 10 minutes following drug administration. Venous plasma samples were obtained prior to drug administration, and then from 15 seconds to 24 hours following drug administration.

Clinical observations and body weights were recorded for all animals at protocol-specified time points. There were no adverse effects noted by clinical observations that could be attributed to the administration of alprazolam. Body weight values showed no remarkable change between treatment sessions.

Mean alprazolam concentration-time profiles in plasma sampled from the left ventricle following either intravenous or inhalation administration of alprazolam to dogs, were, except for the more rapid absorption of the inhaled alprazolam over the first ˜15 seconds, qualitatively similar to each other, as were plasma profiles from the peripheral sampling site following each administration. Mean concentrations increased rapidly to attain Cₘₐₓ at median times ranging from 0.25 minutes to 0.75 minutes, and then declined in an apparent multi-phasic manner. Mean Cₘₐₓ was observed somewhat earlier in left ventricle plasma than in venous plasma, and slightly earlier after inhalation dosing than following intravenous dosing.

Mean Cₘₐₓ for left ventricle plasma was markedly greater (by nearly 7-fold) than for venous plasma for both routes of administration. Inhalation administration resulted in mean Cₘₐₓ values that were approximately 70% of the respective values observed after intravenous dosing, and mean bioavailability estimates for inhalation administration were 85.0% and 95.5%, based on left ventricle plasma and venous plasma, respectively.

Mean terminal elimination half-life estimates were similar for the two routes of administration (approximately 4 minutes in left ventricle plasma and approximately 110 minutes in venous plasma for which samples were taken through much later times).

The mean alprazolam concentration-time profiles for left ventricle plasma were similar following intravenous and inhalation dosing, with concentrations increasing to a maximal value early in the time course, and then declining in a multi-phasic manner. At the first few sampling time points, mean concentrations in plasma from the left ventricle were greater following inhalation administration than after intravenous dosing, but from 0.33 minutes through 3 minutes, concentrations were greater for the intravenous route. At 5 and 10 minutes, mean concentration values were similar for the two routes.

Early in the time course after dosing, mean concentrations were notably higher in left ventricle plasma than in venous plasma. However, by 5 minutes, mean concentrations were comparable in plasma from both sites and for both routes.

Maximum plasma concentration (Cₘₐₓ) was observed somewhat earlier in left ventricle plasma (median times of 0.42 minutes and 0.25 minutes for IV and inhalation dosing, respectively) than in venous plasma (median times of 0.75 minutes and 0.5 minutes for IV and inhalation dosing, respectively), and earlier after inhalation dosing than following intravenous dosing. Concentrations were measurable in left ventricle plasma through the last sampling time (10 minutes) in all cases and were measurable in venous plasma through median times of 240 minutes for IV dosing and 360 minutes for inhalation dosing.

Mean Cₘₐₓ for left ventricle plasma was markedly greater (by nearly 7-fold) than for venous plasma for both routes of administration. Inhalation administration resulted in mean Cₘₐₓ values that were approximately 70% of the respective values observed after intravenous dosing. Mean bioavailability (% F) estimates for inhalation administration, which were based on AUC for left ventricle plasma and for venous plasma, were 85.0% and 95.5%, respectively. The latter values were based on data from three animals, as one animal was replaced prior to administration of the inhalation dose. The bioavailability based upon mean AUC (n=4) by each route was 81.5% for left ventricle sampling and 94.9% for systemic plasma.

Mean terminal elimination half-life (t_{1/2}) estimates were similar for the two routes of administration; approximately 4 minutes in left ventricle plasma and approximately 110 minutes in venous plasma. The difference was due to the much later sampling regime for venous plasma, which allowed for more complete characterization of the terminal phase of the plasma concentration-time curves.

### Frequency and Duration of Treatment Administration

Four animals per session were treated with the test article, alprazolam, in two separate surgical/dosing sessions. The animals were dosed at a level of 0.7 mg of alprazolam in both dosing sessions. For each session, the animals were appropriately anesthetized prior to administration of the test article. Each surgical/dosing session was separated by a washout period of approximately 48 hours from the time of the previous treatment administration.

Session 1: While the animals were anesthetized, the IV test article (3.5 mL of 0.2 mg/mL alprazolam injection) was administered as a 5-second IV bolus via a saphenous vein. Blood sampling commenced at the time of initiation of the bolus (t=0).

Session 2: While the animals were anesthetized, the aerosol generation and administration system was connected in-line to an endotracheal tube to allow for delivery of the aerosol test article (alprazolam coated on a chemical, single-dose heat package). The aerosol resulting from actuation of the alprazolam by the aerosol generation and administration system was administered during a single deep inhalation. Blood sampling commenced at the time of initiation of the deep inhalation (t=0).

### Anesthesia

Anesthesia was performed substantially as described in Example 1.

### Surgical Procedure

The surgical procedure was performed as described in Example 1, with the Blood Sample Collection Schedule as shown in Table 4.

TABLE 4
Blood Sample Collection Schedule

| Session | Site of | |
|---|---|---|
| Number | Collection | Time Points (Time Post t = 0)^{a} |
| 1 | Left | Start of IV dosing, 5 seconds after the start of IV dosing |
| | Ventricle | (i.e., end of IV injection), 10 seconds after start of IV |
| | | dosing, then at 15, 20, 25, 30, 40, 50, 60, 75, 90, 120, 150, and |
| | | 180 seconds post, then at 5 and 10 minutes post t = 0 |
| | Venous | Prior to dose, then at 15, 30, 60, 120, and 180 seconds post, then at |
| | | 5, 10, 20, 30, 60, 120, and 240 minutes post start, and |
| | | 8, 12, 16, and 24 hours post t = 0 |
| 2 | Left | Start of inhalation, 5 seconds into inhalation, 10 seconds |
| | Ventricle | into inhalation, then at 15, 20, 25, 30, 40, 50, 60, 75, 90, |
| | | 120, 150, and 180 seconds post, then at 5 and 10 minutes |
| | | post t = 0 |
| | Venous | Prior to dose, then at 15, 30, 60, 120, and 180 seconds post, |
| | | then at 5, 10, 20, 30, 60 120, and 240 minutes post start, and 8, 12, |
| | | 16, and 24 hours post t = 0 |
| ^{a}The time of the start of IV injection or the time of the start of inhalation was designated as t = 0. A 2-second deviation was allowed on all time points out to and including the 60-second time point. Following this period, deviations for sampling times were determined per Test Facility SOP (±5% of each time point). Collection times were recorded in hour/minute/second format up to and including the 30-minute time point, following which, collection time points | | |
| # were recorded in hour/minute format. Samples were placed on ice after collection until the time of processing. | | |

### Bioanalytical Samples

Whole blood samples were centrifuged. The plasma was extracted and placed in a −70° C. freezer within 90 minutes from the time of collection. All plasma samples were stored until analyzed. Concentrations of alprazolam, and the metabolites α-hydroxyalprazolam and 4-hydroxyalprazolam, in plasma samples were measured using a validated LC-MS/MS method with a limit of quantitation of 1.00 ng/mL for alprazolam, and 300 ng/mL for α-hydroxyalprazolam and 4-hydroxyalprazolam. Plasma samples were mixed with spiking solutions of alprazolam, α-hydroxyalprazolam, 4-hydroxyalprazolam and an internal standard (alprazolam-d₅). Extracts were separated by gradient HPLC on a Phenomenex Synergi Hydro-RP, 4 micron column and subjected to tandem mass spectrometry (MDS Sciex API 3000) with electrospray ionization in positive ion mode and mass ratio monitoring (MRM) detection. Drug or metabolite concentrations were calculated by comparing (drug or metabolite)/internal standard ratios to standard curves prepared in dog plasma.

### Dose Formulation Analysis

Aerosol samples were captured from the aerosol administration system before and after dose administration. These samples were captured in either a single filter to measure Emitted Dose (ED) or in an Anderson Cascade Impactor (ACI) to measure particle size distribution. The analyses confirmed that the aerosol administration system, when loaded with the test article (alprazolam) generated the appropriate aerosol emitted from the dog's endotracheal tube, (i.e., an alprazolam emitted dose of approximately 0.7 mg; (e.g., between 0.61 and 0.76 mg in all test samples' mean pretreatment 0.71 mg and post treatment 0.68 mg; with an aerosol mass median aerodynamic diameter of approximately 2.4 micrometers; between 2.2 and 2.5 micrometers in all test samples' mean pretreatment 2.4 micrometers and post treatment 2.4 micrometers and geometric standard deviation of 2.3).

TABLE 5
Data Summary for Stability of Aerosol

| Test | Before Dosing | | After Dosing | |
|---|---|---|---|---|
| Emitted Dose | 1. 0.72 mg | | 1. 0.69 mg | |
| | 2. 0.76 mg | | 2. 0.74 mg | |
| | 3. 0.65 mg | | 3. 0.61 mg | |
| | Average = 0.71 mg | | Average = 0.68 mg | |
| | SD = 0.05 mg | | SD = 0.05 mg | |
| | MMAD, μm | GSD | MMAD, μm | GSD |
| Aerosol Particle | 2.5 | 2.3 | 2.5 | 2.2 |
| Size Distribution | 2.3 | 2.3 | 2.3 | 2.3 |
| | Ave = 2.4 | Ave = 2.3 | Ave = 2.4 | Ave = 2.3 |

| | | |
|---|---|---|
| Coated Drug | Estazolam = 0.02% | Estazolam = 0.02% |
| Identity and | RRT 1.04 = 0.07% | RRT 1.04 = 0.08% |
| Impurities | RRT 1.31 = 0.48% | RRT 1.31 = 0.69% |
| | RRT 1.52 = 0.51% | RRT 1.52 = 0.56% |
| | RRT 1.65 = 0.13% | RRT 1.65 = 0.29% |
| | RRT 1.83 = 0.46% | RRT 1.83 = 0.44% |
| | Total Impurities = 1.83% | Total Impurities = 2.32% |

MMAD = Mass Medium Aerodynamic Diameter
GSD = Geometric Standard Deviation
RRT = Relative Retention Time

### Pharmacokinetic Analysis Procedures

Individual plasma concentration-time data from four animals, two sampling sites, and two dose routes were analyzed by noncompartmental pharmacokinetic methods via WinNonlin using nominal time points. Areas were calculated using the linear trapezoidal rule. The following pharmacokinetic parameters were derived for individual animals: maximum plasma concentration (Cₘₐₓ), time of maximum plasma concentration (tₘₐₓ); time of last quantifiable plasma concentration (tₗₐₛₜ), area under the concentration versus time curve from time zero to time of last quantifiable plasma concentration (AUCₗₐₛₜ), and apparent terminal elimination half-life (t_{1/2}). A terminal elimination half-life was estimated from a minimum of three concentrations that appeared to be on the terminal elimination portion of the plasma concentration versus time curve. Bioavailability (% F) was calculated from dose normalized AUC values. The resulting individual pharmacokinetic parameter estimates were used to calculate descriptive statistics for the group. Data and descriptive statistics are displayed in tables and figures as appropriate. No additional statistical analysis was performed on the toxicokinetic data.

### Pharmacokinetic

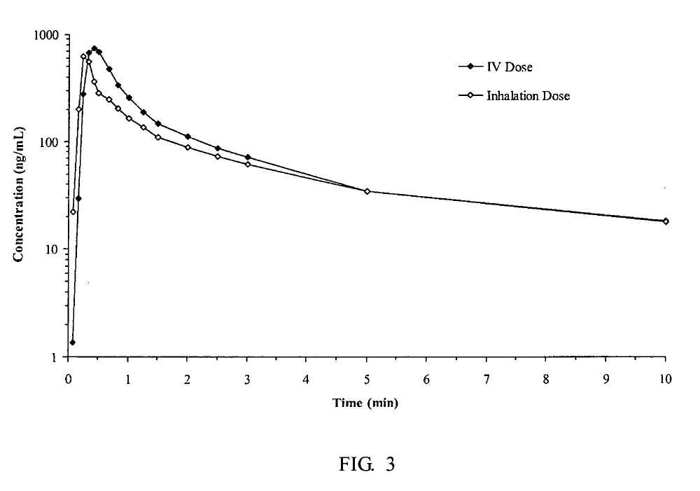

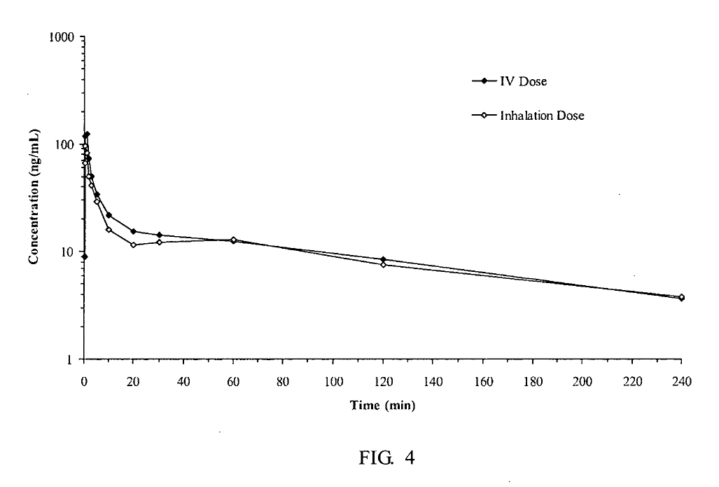

In this comparative pharmacokinetic study, dogs received 0.7 mg of alprazolam as a single deep inhalation of aerosol and an IV bolus in two consecutive treatment sessions. Left ventricular and venous pharmacokinetic parameters of alprazolam were determined and are summarized in Table 6. Concentrations of alprazolam in left ventricular plasma are plotted in FIG. 3. Venous concentrations of alprazolam are plotted in FIG. 4.

The mean alprazolam concentration-time profiles for left ventricle plasma were quite similar following intravenous bolus and inhalation dosing, with concentrations increasing to a maximal value early in the time-course and then declining in a multi-phasic manner. At the first few sampling times, mean concentrations in plasma from the left ventricle were greater following inhalation administration than after intravenous bolus dosing, but from 0.33 min through 3 min, concentrations were greater for the intravenous bolus route. At 5 and 10 min, mean concentrations were similar for the two routes.

The mean alprazolam concentration-time profiles for venous plasma were also quite similar following intravenous and inhalation dosing, with concentrations increasing to a maximal value early in the time-course and then declining in a multi-phasic manner, but with an apparent slight “hump” at 60 min for the inhalation route. At the first sampling time after the start of dosing, for venous plasma, the mean concentration was greater following inhalation administration than after intravenous dosing. At subsequent times through 30 min, mean concentrations were greater for the intravenous route. At later times mean concentrations were similar for the two routes.

Cₘₐₓ was observed somewhat earlier in left ventricle plasma (median times of 0.42 min and, 0.25 min, for IV and inhalation dosing, respectively) than in venous plasma (median times of 0.75 min and 0.5 min, for IV and inhalation dosing, respectively), and earlier after inhalation dosing than following intravenous dosing. Concentrations were measurable in left ventricle plasma through the last sampling time (10 min) in all cases and in venous plasma through median times of 240 min for IV dosing and 360 min for inhalation dosing.

Mean Cₘₐₓ for left ventricle plasma was markedly greater (by nearly 7-fold) than for venous plasma for both routes of administration. Inhalation administration resulted in mean Cₘₐₓ values that were approximately 70% of the respective values observed after intravenous dosing. Mean bioavailability estimates for inhalation administration, which were based on AUC for left ventricle plasma and for venous plasma, were 85.0% and 95.5%, respectively. The bioavailability based upon mean AUC (n=4) by each route is 81.5% for left ventricle sampling and 94.9% for systemic plasma.

Mean terminal elimination half-life estimates were similar for the two routes of administration, approximately 4 min in left ventricle plasma and approximately 110 min in venous plasma. The difference may be due in part to the much later sampling regime for venous plasma, which allowed for more complete characterization of the terminal phase of the plasma concentration-time curves; however, the short t1/2 in the left ventricle also reflects the profound effect of drug redistribution on left ventricular and arterial plasma drug concentrations after drug administration using these methods, which results in the observed rapid drop in these levels over the first few minutes. This rapid drop is clinically useful when a pulsatile drug effect is desired, e.g., for reversing cardiac arrhythmias.

TABLE 6
Summary of Alprazolam Pharmacokinetic Parameters by Treatment

| | | | |
|---|---|---|---|
| | | Aerosol | IV Bolus |
| Parameter | Units | Mean^{a} ± SD | Mean^{a} ± SD |

Pharmacokinetic Parameters from Venous Plasma Concentrations

| | | | |
|---|---|---|---|
| Cₘₐₓ | ng/mL | 102 ± 57.2 | 147 ± 93.5 |
| Tₘₐₓ | Min | 0.5 | 0.75 |
| Tₗₐₛₜ | Hr | 360 | 240 |
| AUC₀₋₂₄₀ | ng min/mL | 2300 ± 232 | 2540 ± 497 |
| AUCₗₐₛₜ | ng min/mL | 2600 ± 484 | 2720 ± 828 |
| AUC | ng min/mL | 2980 ± 292 | 3140 ± 651 |
| Half-Life | Min | 115 ± 12.9 | 106 ± 22 |
| F^{b} | % | 95.5 ± 22 | NA |

Pharmacokinetic Parameters from Left Ventricular Plasma

| | | | |
|---|---|---|---|
| Cₘₐₓ | ng/mL | 676 ± 99.5 | 967 ± 303 |
| Tₘₐₓ | min | 0.25 | 0.42 |

^{a}Median for tₘₐₓ and tₗₐₛₜ; n = 4
^{b}n = 3

### Example 3

### General Procedure for Determining Whether a Drug is “Heat Stable”

Drug is dissolved or suspended in a solvent (e.g., dichloromethane or methanol). The solution or suspension is coated to about a 4 micron thickness on a stainless steel substrate of about 8 cm² surface area. The substrate may either be a standard stainless steel foil or a heat-passivated stainless steel foil. The substrate is heated to a temperature sufficient to generate a thermal vapor (generally ˜350° C.) but at least to a temperature of 200° C. with an air flow typically of 20 L/min (1 m/s) passing over the film during heating. The heating is done in a volatilization chamber fitted with a trap (such as described above).

After vaporization is complete, airflow is discontinued and the resultant aerosol is analyzed for purity using the methods disclosed herein. If the resultant aerosol contains less than 10% drug degradation product, i.e., the TSR≧9, then the drug is a heat stable drug. If, however, at about 4 micron thickness, greater than 10% degradation is determined, the experiment is repeated at the same conditions, except that film thicknesses of about 1.5 microns, and of about 0.5 micron, respectively, are used. If a decrease in degradation products relative to the 4 micron thickness is seen at either of these thinner film thicknesses, a plot of film thickness versus purity is graphed and extrapolated out to a film thickness of 0.05 microns. The graph is used to determine if there exists a film thickness where the purity of the aerosol would be such that it contains less than 10% drug degradation products. If such a point exists on the graph, then the drug is defined as a heat stable drug.

A number of embodiments of the invention have been described. Nevertheless, it will be understood that various modifications may be made without departing from the spirit and scope of the invention. Accordingly, other embodiments are within the scope of the following claims.

What is claimed is:

1. A method of identifying a cardiovascular safe dose of a drug active agent for delivery by inhalation, the method comprising:
(a) determining a peak arterial concentration of the drug active agent following
(i) inhalation delivery of the drug active agent, and
(ii) intravenous delivery of the drug active agent,
(b) identifying a cardiovascular safe intravenous dose of the drug active agent based on cardiovascular safety measurements taken after intravenous delivery of the drug active agent; and
(c) defining a cardiovascular safe inhaled dose of the drug active agent as less than or equal to the cardiovascular safe intravenous dose divided by the ratio of the peak drug active agent concentration produced by the inhalation delivery relative to that produced by the intravenous delivery.

2. A method of identifying a cardiovascular safe dose of a drug active agent for systemic delivery by inhalation, the method comprising:
(a) determining a rate of absorption of the drug active agent into the arterial circulation by inhalation delivery, and
(b) determining the cardiovascular safety of the drug active agent delivered at a substantially identical rate at one or more doses by intravenous delivery,
wherein the cardiovascular safe dose of the inhaled active agent is equal to or less than the dose that is determined to be safe when delivered at a substantially identical rate by the intravenous delivery.

3. An aerosol-releasing device for inhalation therapy, wherein the device releases one or more doses of aerosol that, when inhaled by a mammal, results in a spike index of between about 1.5 and 10, wherein the spike index is determined by:
(a) administering to a subject an equal amount of a drug active agent by both inhalation delivery and intravenous delivery;
(b) identifying an inhaled peak arterial plasma concentration after delivery of drug active agent in an aerosol by inhalation;
(c) identifying an intravenous peak arterial plasma concentration after delivery of a substantially identical dose of the drug active agent by IV injection; and
(d) dividing the inhaled peak arterial plasma concentration by the intravenous peak arterial plasma concentration to determine the spike index.

4. A method of delivering a drug active agent to a mammal, the method comprising administering the drug active agent by inhalation in the form of an aerosol, wherein the administration produces a spike index between 2 and 6, and wherein the peak left ventricular plasma concentration of the drug active agent is achieved in less than 30 seconds.

5. The method of claim 1, further comprising:
(a) identifying an effective intravenous dose of the drug active agent that produces a desirable response in a mammal,
(b) defining an effective inhalation dose by dividing the effective intravenous dose by the ratio of the peak active agent concentration produced by inhalation relative to that produced by the intravenous delivery to yield an effective inhalation dose, wherein the effective inhalation dose is less than the safe inhalation dose; and
(c) selecting a therapeutic inhalation dose of the drug active agent, wherein the therapeutic inhalation dose is less than or equal to the safe inhalation dose and greater than or equal to the effective inhalation dose.

6. The method of claim 1, wherein the drug active agent comprises a drug selected from the group consisting of acebutolol, acetaminophen, alprazolam, amantadine, amitriptyline, apomorphine diacetate, apomorphine hydrochloride, atropine, azatadine, betahistine, brompheniramine, bumetanide, buprenorphine, bupropion hydrochloride, butalbital, butorphanol, carbinoxamine maleate, celecoxib, chlordiazepoxide, chlorpheniramine, chlorzoxazone, ciclesonide, citalopram, clomipramine, clonazepam, clozapine, codeine, cyclobenzaprine, cyproheptadine, dapsone, diazepam, diclofenac ethyl ester, diflunisal, disopyramide, doxepin, estradiol, ephedrine, estazolam, ethacrynic acid, fenfluramine, fenoprofen, flecainide, flunitrazepam, galanthamine, granisetron, haloperidol, hydromorphone, hydroxychloroquine, ibuprofen, imipramine, indomethacin ethyl ester, indomethacin methyl ester, isocarboxazid, ketamine, ketoprofen, ketoprofen ethyl ester, ketoprofen methyl ester, ketorolac ethyl ester, ketorolac methyl ester, ketotifen, lamotrigine, lidocaine, loperamide, loratadine, loxapine, maprotiline, memantine, meperidine, metaproterenol, methoxsalen, metoprolol, mexiletine HCl, midazolam, mirtazapine, morphine, nalbuphine, naloxone, naproxen, naratriptan, nortriptyline, olanzapine, orphenadrine, oxycodone, paroxetine, pergolide, phenytoin, pindolol, piribedil, pramipexole, procainamide, prochloperazine, propafenone, propranolol, pyrilamine, quetiapine, quinidine, rizatriptan, ropinirole, sertraline, selegiline, sildenafil, spironolactone, tacrine, tadalafil, terbutaline, testosterone, thalidomide, theophylline, tocainide, toremifene, trazodone, triazolam, trifluoperazine, valproic acid, venlafaxine, vitamin E, zaleplon, zotepine, amoxapine, atenolol, benztropine, caffeine, doxylamine, estradiol 17-acetate, flurazepam, flurbiprofen, hydroxyzine, ibutilide, indomethacin norcholine ester, ketorolac norcholine ester, melatonin, metoclopramide, nabumetone, perphenazine, protriptyline HCl, quinine, triamterene, trimipramine, zonisamide, bergapten, chlorpromazine, colchicine, diltiazem, donepezil, eletriptan, estradiol-3,17-diacetate, efavirenz, esmolol, fentanyl, flunisolide, fluoxetine, hyoscyamine, indomethacin, isotretinoin, linezolid, meclizine, paracoxib, pioglitazone, rofecoxib, sumatriptan, tolterodine, tramadol, tranylcypromine, trimipramine maleate, valdecoxib, vardenafil, verapamil, zolmitriptan, zolpidem, zopiclone, bromazepam, buspirone, cinnarizine, dipyridamole, naltrexone, sotalol, telmisartan, temazepam, albuterol, apomorphine hydrochloride diacetate, carbinoxamine, clonidine, diphenhydramine, thambutol, fluticasone proprionate, fluconazole, lovastatin, lorazepam N,O-diacetyl, methadone, nefazodone, oxybutynin, promazine, promethazine, sibutramine, tamoxifen, tolfenamic acid, aripiprazole, astemizole, benazepril, clemastine, estradiol 17-heptanoate, fluphenazine, protriptyline, ethambutal, frovatriptan, pyrilamine maleate, scopolamine, and triamcinolene acetonide.

7. The method of claim 1, wherein the inhaled drug active agent comprises aerosol particles produced by drug heating and vaporization characterized by an MMAD of 1-3 μm less than 5% drug degradation products by weight.

8. The method of claim 1, wherein drug active agent comprises a drug selected from the group of anesthetics, anticonvulsants, antidepressants, antidiabetic agents, antidotes, antiemetics, antihistamines, anti-infective agents, antineoplastics, antiparkisonian drugs, antirheumatic agents, antipsychotics, anxiolytics, appetite stimulants and suppressants, blood modifiers, cardiovascular agents, central nervous system stimulants, drugs for Alzheimer's disease management, drugs for cystic fibrosis management, diagnostics, dietary supplements, drugs for erectile dysfunction, gastrointestinal agents, hormones, drugs for the treatment of alcoholism, drugs for the treatment of addiction, immunosuppressives, mast cell stabilizers, migraine preparations, motion sickness products, drugs for multiple sclerosis management, muscle relaxants, nonsteroidal anti-inflammatories, opioids, other analgesics, stimulants, opthalmic preparations, osteoporosis preparations, prostaglandins, respiratory agents, sedatives and hypnotics, skin and mucous membrane agents, smoking cessation aids, Tourette's syndrome agents, urinary tract agents, and vertigo agents.

9. An inhalation device comprising a dose of a drug active agent identified by the methods of claim 1.

10. A method of delivering a heat stable drug active agent to a mammal to achieve a rapid therapeutic effect, the method comprising
generating an aerosol of the drug active agent, and
delivering the aerosol into the pulmonary tract of the mammal to produce a peak arterial plasma concentration, wherein the peak plasma concentration is achieved more rapidly than following intravenous bolus delivery of the same medication.

11. The method of claim 10, wherein the peak plasma concentration of the drug active agent following inhalation is between 0.5 times and 1.5 times the peak plasma concentration following intravenous bolus delivery.

12. A method of delivering a heat stable drug active agent to a mammal, the method comprising
generating an aerosol of the medication, and
delivering the aerosol into the pulmonary tract of the mammal to produce (a) a peak arterial plasma concentration and (b) a peak venous plasma concentration, wherein the peak arterial plasma concentration is between 2 and 10 times greater than the peak venous plasma concentration.
